SEQUENCE LISTING (48 sequences)

SEQ ID NO: 1 - protein, 119 aa, Mus musculus
QVQLVQPGAEVVKPGASVKLSCKTSGYTFTSNWMHWVKQRPGQGLEWIGEIDPSDSYTNYNQNFKGKAKLTVDKSTSTAYMEVSSLRSDDTAVYYCARGSNPYYYAMDYWGQGTSVTVS

SEQ ID NO: 2 - protein, 119 aa, Mus musculus
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVS

SEQ ID NO: 3 - protein, 121 aa, Mus musculus
QVQLLESGAELVRPGSSVKISCKASGYAFSSYWMNWVKQRPGQGLEWIGQIWPGDGDTNYNGKFKGKATLTADESSSTAYMQLSSLRSEDSAVYSCARRETTTVGRYYYAMDYWGQGTTVT

SEQ ID NO: 4 - protein, 123 aa, Mus musculus
QVQLQQSGAELVRPGSSVKISCKASGYAFSSYWMNWVKQRPGQGLEWIGQIWPGDGDTNYNGKFKGKATLTADESSSTAYMQLSSLASEDSAVYFCARRETTTVGRYYYAMDYWGQGTSVTVS

SEQ ID NO: 5 - protein, 120 aa, Mus musculus
EVQLQQSGPELIKPGASVKMSCKASGYTFTSYVMHWVKQKPGQGLEWIGYINPYNDGTKYNEKFKGKATLTSDKSSSTAYMELSSLTSEDSAVYYCARGTYYYGSRVFDYWGQGTTLTVS

SEQ ID NO: 6 - protein, 119 aa, Mus musculus
EVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVS

SEQ ID NO: 7 - protein, 104 aa, Mus musculus
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 8 - protein, 104 aa, Mus musculus; identical to SEQ ID NO: 7

SEQ ID NO: 9 - protein, 111 aa, Mus musculus
ELVLTQSPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 10 - protein, 111 aa, Mus musculus
DILLTQTPASLAVSLGQRATISCKASQSVDYDGDSYLNWYQQIPGQPPKLLIYDASNLVSGIPPRFSGSGSGTDFTLNIHPVEKVDAATYHCQQSTEDPWTFGGGTKLEIK

SEQ ID NO: 11 - protein, 112 aa, Mus musculus
DIVMTQAAPSIPVTPGESVSISCRSSKSLLNSNGNTYLYWFLQRPGQSPQLLIYRMSNLASGVPDRFSGSGSGTAFTLRISRVEAEDVGVYYCMQHLEYPFTFGAGTKLELK

SEQ ID NO: 12 - protein, 107 aa, Mus musculus
DIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEIT

SEQ ID NO: 13 - protein, 449 aa, Artificial Sequence
  Humanised Mus musculus
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 14 - protein, 211 aa, Artificial Sequence
  Humanised Mus musculus
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 15 - protein, 447 aa, Mus musculus
EVQLVESGGGLVQPGGSLRLSCAASGYTFSSYWIEWVRQAPGKGLEWIGEILPGGGDTNYNEIFKGRATFSADTSKNTAYLQMNSLRAEDTAVYYCTRRVPIRLDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 16 - protein, 213 aa, Mus musculus
DIQLTQSPSSLSASVGDRVTITCKASQSVDYEGDSFLNWYQQKPGKAPKLLIYAASNLESGVPSRFSGSGSGTDFTLTISSLQPEDFATYYCQQSNEDPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSF

SEQ ID NO: 17 - protein, 117 aa, Mus musculus [sequence omitted]

SEQ ID NO: 18 - protein, 111 aa, Mus musculus [sequence omitted]

SEQ ID NO: 19 - protein, 5 aa, Mus musculus
SYWIE

SEQ ID NO: 20 - protein, 17 aa, Mus musculus
EILPGGGDTNYNEIFKG

SEQ ID NO: 21 - protein, 8 aa, Mus musculus
RVPIRLDY

SEQ ID NO: 22 - protein, 15 aa, Mus musculus
KASQSVDYEGDSFLN

SEQ ID NO: 23 - protein, 7 aa, Mus musculus
AASNLES

SEQ ID NO: 24 - protein, 9 aa, Mus musculus
QQSNEDPLT

SEQ ID NO: 25 - protein, 127 aa, Mus musculus
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 26 - protein, 119 aa, Mus musculus
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 27 - protein, 5 aa, Mus musculus
RYIIN

SEQ ID NO: 28 - protein, 17 aa, Mus musculus
RIIPILGVENYAQKFQG

SEQ ID NO: 29 - protein, 6 aa, Mus musculus
KDWFDY

SEQ ID NO: 30 - protein, 11 aa, Mus musculus
RASQSVSSYLA

SEQ ID NO: 31 - protein, 7 aa, Mus musculus
GASSRAT

SEQ ID NO: 32 - protein, 9 aa, Mus musculus
QQYGSSPLT

SEQ ID NO: 33 - protein, 109 aa, Mus musculus
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQG

SEQ ID NO: 34 - protein, 106 aa, Mus musculus
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQG

SEQ ID NO: 35 - protein, 9 aa, Mus musculus
YTFTSYWLH

SEQ ID NO: 36 - protein, 14 aa, Mus musculus
WIGYINPRNDYTEY

SEQ ID NO: 37 - protein, 8 aa, Mus musculus
RRDITTFY

SEQ ID NO: 38 - protein, 14 aa, Mus musculus
QSVLYSANHKNYLA

SEQ ID NO: 39 - protein, 11 aa, Mus musculus
LLIYWASTRES

SEQ ID NO: 40 - protein, 7 aa, Mus musculus
HQYLSSW

SEQ ID NO: 41 - protein, 445 aa, Mus musculus
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 42 - protein, 219 aa, Mus musculus
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 43 - protein, 329 aa, Mus musculus
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 44 - protein, 329 aa, Mus musculus
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 45 - protein, 105 aa, Mus musculus [sequence omitted]

SEQ ID NO: 46 - protein, 105 aa, Mus musculus
VAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 47 - protein, 103 aa, Mus musculus
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 48 - protein, 103 aa, Mus musculus
KAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTESS

(19) United States
(12) Patent Application Publication
(10) Pub. No.: US 2025/0352658 A1
(43) Pub. Date: Nov. 20, 2025

(54) COMBINATION THERAPY USING ANTIBODY-DRUG CONJUGATES

(71) Applicants: ADC THERAPEUTICS SA, Epalinges (CH); MEDIMMUNE LIMITED, Cambridge, Cambridgeshire (GB)

(72) Inventors: Patricius Hendrikus Cornelis VAN BERKEL, London (GB); Francesca ZAMMARCHI, London (GB)

(21) Appl. No.: 18/572,300

(22) PCT Filed: Jun. 27, 2022
(86) PCT No.: PCT/EP2022/067603

§ 371 Date: Dec. 20, 2023

(30) Foreign Application Priority Data
Jun. 29, 2021 (GB) 2109373.7
Jun. 29, 2021 (GB) 2109375.2
Jun. 29, 2021 (GB) 2109377.8

(51) Int. Cl. A61K 47/68; A61K 39/00; A61K 39/395; A61P 35/00

(52) U.S. Cl. CPC A61K 47/68035; A61K 39/39558; A61K 47/6851; A61P 35/00; A61K 2039/505

(57) ABSTRACT

The present disclosure relates to combinations of anti-CD19 antibody drug conjugates and anti-CD79b conjugates, and their use in therapy, such as treating proliferative disorders.

### EARLIER APPLICATIONS

This application claims priority from United Kingdom application numbers GB2109373.7, filed 29 Jun. 2021; GB2109375.2, filed 29 Jun. 2021; and GB2109377.8, filed 29 Jun. 2021. The priority applications are hereby incorporated by reference in their entirety and for any and all purposes as if fully set forth herein.

### REFERENCE TO SEQUENCE LISTING SUBMITTED ELECTRONICALLY

The contents of the electronic sequence listing (0233-0029US1_SL.xml; Size: 48 KB; and Date of Creation Dec. 12, 2023) submitted herewith, are herein incorporated by reference in their entirety.

### FIELD

The present disclosure relates to combination therapies for the treatment of pathological conditions, such as cancer. In particular, the present disclosure relates to combination therapies comprising treatment with an anti-CD19 Antibody Drug Conjugate (anti-CD19 ADC) and an anti-CD79b agent.

### BACKGROUND

### Antibody Therapy

Antibody therapy has been established for the targeted treatment of subjects with cancer, immunological and angiogenic disorders. The use of antibody-drug conjugates (ADC), i.e. immunoconjugates, for the local delivery of cytotoxic or cytostatic agents, i.e. drugs to kill or inhibit tumour cells in the treatment of cancer, targets delivery of the drug moiety to tumours, and intracellular accumulation therein, whereas systemic administration of these unconjugated drug agents may result in unacceptable levels of toxicity to normal cells.

### CD19

CD19 is a 95 kDa membrane receptor that is expressed early in B cell differentiation and continues to be expressed until the B cells are triggered to terminally differentiate. The CD19 extracellular domain contains two C2-type immunoglobulin (IG)-like domains separated by a smaller potentially disulfide-linked domain. The CD19 cytoplasmic domain is structurally unique, but highly conserved between human, mouse, and guinea pig. CD19 is part of a protein complex found on the cell surface of B-lymphocytes. The protein complex includes CD19, CD21 (complement receptor, type 2), CD81 (TAPA-1), and CD225 (Leu-13)

CD19 is an important regulator of transmembrane signals in B cells. An increase or decrease in the cell surface density of CD19 affects B cell development and function, resulting in diseases such as autoimmunity or hypogammaglobulinemia. The CD19 complex potentiates the response of B cells to antigen in vivo through cross-linking of two separate signal transduction complexes found on B cell membranes. The two signal transduction complexes, associated with membrane IgM and CD19, activate phospholipase C (PLC) by different mechanisms. CD19 and B cell receptor cross-linking reduces the number of IgM molecules required to activate PLC. CD19 also functions as a specialized adapter protein for the amplification of Arc family kinases.

CD19 binding has been shown to both enhance and inhibit B-cell activation and proliferation, depending on the amount of cross-linking that occurs. CD19 is expressed on greater than 90% of B-cell lymphomas and has been predicted to affect growth of lymphomas in vitro and in vivo.

### Therapeutic Uses of Anti-CD19 ADCs

The efficacy of an Antibody Drug Conjugate comprising an anti-CD19 antibody (an anti-CD19-ADC) in the treatment of, for example, cancer has been established-see, for example, WO2014/057117 and WO2016/166298.

Research continues to further improve the efficacy, tolerability, and clinical utility of anti-CD19 ADCs. To this end, the present authors have identified clinically advantageous combination therapies in which an anti-CD19 ADC is administered in combination with at least one anti-CD79b agent.

### SUMMARY

The present authors have determined that the administration of a combination of an anti-CD19 ADC and an anti-CD79b agent to an individual leads to unexpected clinical advantages. The present authors have further determined that administration of an anti-CD19 ADC to an individual that has either been treated with, or is being treated with, and an anti-CD79b agent leads to a synergistic increase in treatment efficacy.

Accordingly, in a first aspect the present disclosure provides a method of selecting an individual as suitable for treatment with an anti-CD19 ADC, wherein the individual is selected for treatment with the anti-CD19 ADC if the individual has been treated, or is being treated, with an anti-CD79b agent. The individual may be selected for treatment if the individual is refractory to treatment, or further treatment, with the anti-CD79b agent.

In another aspect, the present disclosure provides a method for treating a disorder in an individual, the method comprising selecting an individual as suitable for treatment by a method of the first aspect, and then administering to the individual an effective amount of the anti-CD19 ADC. The method of treatment may further comprise administering an anti-CD79b agent in combination with the anti-CD19 ADC.

In another aspect the disclosure provides a method for treating a disorder in an individual, the method comprising administering to the individual an effective amount of an anti-CD19 ADC and an anti-CD79b agent. The individual may be selected for treatment according to a method according of the first aspect.

The disorder may be a proliferative disease, for example a cancer such as non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

The anti-CD19 ADC may be Loncastuximab tesirine.

The anti-CD19 ADC may be ADCx19 described herein.

The anti-CD79b agent may be Polatuzumab vedotin.

The individual may be human. The individual may have cancer, or may have been determined to have cancer. The individual may have, or have been determined to have, a CD19+ cancer or CD19+ tumour-associated non-tumour cells, such as CD19+ infiltrating B-cells.

In the disclosed methods the anti-CD19 ADC may be administered before the anti-CD79b agent, simultaneous with the anti-CD79b agent, or after the anti-CD79b agent. The disclosed methods may comprise administering a further chemotherapeutic agent to the individual.

In another aspect, the present disclosure provides an anti-CD19 ADC, or a composition comprising an anti-CD19 ADC, for use in a method of treatment as described herein.

In one aspect, the present disclosure provides an anti-CD79b agent, or a composition comprising an anti-CD79b agent, for use in a method of treatment as described herein.

In a further aspect, the present disclosure provides for the use of an anti-CD19 ADC or an anti-CD79b agent in the manufacture of a medicament for treating a disorder in an individual, wherein the treatment comprises a method of treatment as described herein.

In another aspect, the disclosure provides a first composition comprising an anti-CD19 ADC for use in a method of treating a disorder in an individual, wherein the treatment comprises administration of the first composition in combination with a second composition comprising an anti-CD79b agent.

Also provided by this aspect is a first composition comprising an anti-CD79b agent for use in a method of treating a disorder in an individual, wherein the treatment comprises administration of the first composition in combination with a second composition comprising an anti-CD19 ADC.

The disorder may be a proliferative disease, for example a cancer such as non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

The anti-CD19 ADC may be Loncastuximab tesirine.

The anti-CD19 ADC may be ADCx19 described herein.

The anti-CD79b agent may be Polatuzumab vedotin.

The individual may be human. The individual may have cancer, or may have been determined to have cancer. The individual may have, or have been determined to have, a CD19+ cancer or CD19+ tumour-associated non-tumour cells, such as CD19+ infiltrating B-cells.

The first composition may be administered before the second composition, simultaneous with the second composition, or after the second composition. The treatment may comprise administering a further chemotherapeutic agent to the individual.

In a further aspect, the disclosure provides the use of an anti-CD19 ADC in the manufacture of a medicament for treating a disorder in an individual, wherein the medicament comprises an anti-CD19 ADC, and wherein the treatment comprises administration of the medicament in combination with a composition comprising anti-CD79b agent.

Also provided by this aspect is the use of anti-CD79b agent in the manufacture of a medicament for treating a disorder in an individual, wherein the medicament comprises an anti-CD79b agent, and wherein the treatment comprises administration of the medicament in combination with a composition comprising an anti-CD19 ADC.

The disorder may be a proliferative disease, for example a cancer such as non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

The anti-CD19 ADC may be Loncastuximab tesirine.

The anti-CD19 ADC may be ADCx19 described herein.

The anti-CD79b agent may be Polatuzumab vedotin.

The individual may be human. The individual may have cancer, or may have been determined to have cancer. The individual may have, or have been determined to have, a CD19+ cancer or CD19+ tumour-associated non-tumour cells, such as CD19+ infiltrating B-cells.

The medicament may be administered before the composition, simultaneous with the composition, or after the composition. The treatment may comprise administering a further chemotherapeutic agent to the individual.

Another aspect of the disclosure provides a kit comprising:
a first medicament comprising an anti-CD19 ADC;
a package insert comprising instructions for administration of the first medicament according to a method of treatment as disclosed herein. The kit may further comprise a second medicament comprising an anti-CD79b agent.

Another aspect of the disclosure provides a kit comprising:
a first medicament comprising an anti-CD19 ADC;
a second medicament comprising an anti-CD79b agent; and, optionally,
a package insert comprising instructions for administration of the first medicament to an individual in combination with the second medicament for the treatment of a disorder.

Also provided by this aspect is a kit comprising a medicament comprising an anti-CD19 ADC and a package insert comprising instructions for administration of the medicament to an individual in combination with a composition comprising an anti-CD79b agent for the treatment of a disorder.

Further provided by this aspect is a kit comprising a medicament comprising an anti-CD79b agent and a package insert comprising instructions for administration of the medicament to an individual in combination with a composition comprising an anti-CD19 ADC for the treatment of a disorder.

The disorder may be a proliferative disease, for example a cancer such as non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

The anti-CD19 ADC may be Loncastuximab tesirine.

The anti-CD19 ADC may be ADCx19 described herein.

The anti-CD79b agent may be Polatuzumab vedotin.

The individual may be human. The individual may have cancer, or may have been determined to have cancer. The individual may have, or have been determined to have, a CD19+ cancer or CD19+ tumour-associated non-tumour cells, such as CD19+ infiltrating B-cells.

The medicament or composition comprising the anti-CD19 ADC may be administered before the medicament or composition comprising the anti-CD79b agent, simultaneous with the medicament or composition comprising the anti-CD79b agent, or after the medicament or composition comprising the anti-CD79b agent. The treatment may comprise administering a further chemotherapeutic agent to the individual.

In a yet further aspect, the disclosure provides a composition comprising an anti-CD19 ADC and an anti-CD79b agent, along with the use of such composition in the methods disclosed herein.

Also provided in this aspect of the disclosure is a method of treating a disorder in an individual, the method comprising administering to the individual an effective amount of the composition comprising an anti-CD19 ADC and an anti-CD79b agent.

Also provided in this aspect of the disclosure is a composition comprising an anti-CD19 ADC and an anti-CD79b agent for use in a method of treating a disorder in an individual.

Also provided in this aspect of the disclosure is the use of a composition comprising an anti-CD19 ADC and an anti-CD79b agent in the manufacture of a medicament for treating a disorder in an individual.

Also provided in this aspect of the disclosure is a kit comprising composition comprising an anti-CD19 ADC and an anti-CD79b agent and a set of instructions for administration of the medicament to an individual for the treatment of a disorder.

The disorder may be a proliferative disease, for example a cancer such as non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

The anti-CD19-ADC may be ADCX19 as described herein.

The anti-CD79b agent may be Polatuzumab vedotin.

The individual may be human. The individual may have cancer, or may have been determined to have cancer. The individual may have, or have been determined to have, a CD19+ cancer or CD19+ tumour-associated non-tumour cells, such as CD19+ infiltrating B-cells.

The treatment may comprise administering a further chemotherapeutic agent to the individual.

The present disclosure also relates more generally to a combination of an antibody drug conjugate that comprises a PBD dimer as the warhead (such as the PBDs described herein) and an antibody drug conjugate that comprises monomethyl auristatin E as the warhead, and therapeutics methods as described herein which comprise administering a combination of the two antibody drug conjugates. The antibodies for each of the individual agents are selected to bind to a different target molecule. One example of an antibody drug conjugate that comprises monomethyl auristatin E is where the antibody targets anti-CD79b. Examples of antibody drug conjugates that comprise a PBD dimer, are those that target CD19 as described above as well as those that target CD25 or CD22, as described below.

Accordingly, in a further aspect the present disclosure provides a combination of (i) an antibody drug conjugate that comprises a PBD dimer; and (ii) an antibody drug conjugate that comprises monomethyl auristatin E, wherein the antibody of (i) binds to a different target molecule to the antibody of (ii) (e.g. different cell surface molecule such as proteins, receptors and the like).

In a related aspect the present disclosure provides a method of treating an individual suffering from a proliferative disorder by administering to the individual a combination of (i) an antibody drug conjugate that comprises a PBD dimer; and (ii) an antibody drug conjugate that comprises monomethyl auristatin E, wherein the antibody of (i) binds to a different target molecule to the antibody of (ii) (e.g. different cell surface molecule such as proteins, receptors and the like). Such administration may be at the same time or separately.

It will be appreciated by a person skilled in the art that further embodiments described below in detail for anti-CD19 ADCs and anti-CD79b ADCs, such as the selection of PBD, disorders to be treated, patient selection and administration, can be applied mutatis mutandis to the ADCs of (i) and (ii) that bind to different targets. Particular examples of other targets are CD25 and CD22 which are described in more detail below.

### CD25

The type I transmembrane protein CD25 is present on activated T- and B-cells, some thymocytes, myeloid precursors, and oligodendrocytes. On activated T-cells, it forms heterodimers with the beta- and gamma subunits (CD122 and CD132), thus comprising the high-affinity receptor for IL-2. This ligand represents a survival factor for activated T-cells, as removal of IL-2 leads to immediate death of these cells.

In case of B-cells, CD25 is physiologically expressed in early developmental stages of late pro-B and pre-B cells. Malignancies arising from this stage of B-cell differentiation may thus also express CD25. Mast cell lesions are also positive for CD25 which is thus considered as a key diagnostic criterion for determination of systemic mastocytosis. In Hodgkin lymphomas, CD25 is reported to be not expressed in Hodgkin-/Reed-Sternberg cells in nodular lymphocyte predominance Hodgkin lymphoma (NLPHL), whereas the same cell type expresses CD25 at varying levels in classical Hodgkin' lymphomas of mixed cellularity type. The general expression levels are reported to be lower than in tumor infiltrating lymphocytes (TILs), which may result in problems demonstrating CD25 tumor cells in these cases (Levi et al., Merz et al, 1995).

Expression of the target antigen has also been reported for several B- and T-cell-derived subtypes of non-Hodgkin-lymphomas, i.e. B-cell chronic lymphatic leukemia, hairy cell leukemia, small cell lymphocytic lymphoma/chronic lymphocytic leukemia as well as adult T-cell leukemia/lymphoma and anaplastic large cell lymphoma.

CD25 may be localised to the membrane, with some expression observed in the cytoplasm. Soluble CD25 may also be observed outside of cells, such as in serum.

### Therapeutic Uses of Anti-CD25 ADCs

The efficacy of an Antibody Drug Conjugate comprising an anti-CD25 antibody (an anti CD25-ADC) in the treatment of, for example, cancer has been established-see, for example, WO2014/057119, WO2016/083468, WO2016/166341, and WO2019/224275.

Research continues to further improve the efficacy, tolerability, and clinical utility of anti-CD25 ADCs. To this end, the present authors have identified clinically advantageous combination therapies in which an anti-CD25 ADC is administered in combination with at least one anti-CD79b agent.

Thus, in other aspects of the invention, instead of an anti-CD19-ADC in combination with an Anti-CD76b agent, an anti-CD25-ADC is used in combination with an anti-CD79b agent. Therefore all references to “anti-CD19-ADC” in the above embodiments can in additional embodiments be replaced with “anti-CD25-ADC”.

### CD22

CD22 is a 135-kDa type I transmembrane sialoglycoprotein of the immunoglobulin (lg) superfamily. CD22 expression is specific to B cells and is developmentally regulated so that expression is limited in pro-B and pre-B cells. As B-cells mature, expression increases and localization of CD22 shifts to the cell surface. CD22 is strongly expressed on follicular, mantle and marginal-zone B cells, but is weakly present in germinal B cells. CD22 is an inhibitory co-receptor that down modulates B-cell receptor (BCR) signalling by setting a signalling threshold that prevents overstimulation of B cells.

Antibodies against CD22, such as epratuzumab (hLL2), have been used for treatment of a variety of cancers and autoimmune diseases, including but not limited to acute lymphoblastic leukemia, chronic lymphocytic leukemia, non-Hodgkin's lymphoma, follicular lymphoma, diffuse large B-cell lymphoma, mantle cell lymphoma, systemic lupus erythematosus, and primary Sjögren's syndrome. A phase III clinical trial of epratuzumab in systemic lupus erythematosus is currently in progress (see, e.g., ClinicalTrials.gov, “Study of Epratuzumab versus Placebo in Subjects with Moderate to Severe General Systemic Lupus Erythematosus (EMBODY 1)”). Because CD22 regulates B-cell functions and survival, it is an important link for modulating humoral immunity and proliferation of B-cell lymphomas and a target for therapeutic antibodies in cancer and autoimmune disease.

### Therapeutic Uses of Anti-CD22 ADCs

The efficacy of an Antibody Drug Conjugate comprising an anti-CD22 antibody (an anti-CD22-ADC) in the treatment of, for example, cancer has been established-see, for example, WO2014/057122 and WO2016/166307, or as described in Kantarjian et al., (2016, New Eng J Med).

Research continues to further improve the efficacy, tolerability, and clinical utility of anti-CD22 ADCs. To this end, the present authors have identified clinically advantageous combination therapies in which an anti-CD22 ADC is administered in combination with at least one anti-CD79b agent.

Thus, in other aspects of the invention, instead of an anti-CD19-ADC in combination with an anti-CD79b agent, an anti-CD22-ADC is used in combination with an anti-CD79b agent. Therefore all references to “anti-CD19-ADC” in the above embodiments can in additional embodiments be replaced with “anti-CD22-ADC”.

### DETAILED DESCRIPTION

### Antibody Drug Conjugates (ADCs)

The present disclosure relates to the improved efficacy of combinations of an ADC and an anti-CD79b agent.

The ADC can deliver a drug to a target location. The target location is preferably a proliferative cell population. The antibody is an antibody for an antigen present on a proliferative cell population. In one aspect the antigen is absent or present at a reduced level in a non-proliferative cell population compared to the amount of antigen present in the proliferative cell population, for example a tumour cell population.

The ADC may comprise a linker which may be cleaved so as to release the drug at the target location. The drug may be a compound selected from RelA, RelB, RelC, RelD or RelE. Thus, the conjugate may be used to selectively provide a compound RelA, RelB, Rel C, RelD or RelE to the target location.

The linker may be cleaved by an enzyme present at the target location.

The disclosure particularly relates to treatment with an anti-CD19 ADC disclosed in WO2014/057117, and as herein described.

### Anti-CD19 ADCs

As used herein, the terms “anti-CD19 ADC” or “CD19-ADC” refers to an ADC in which the antibody component is an anti-CD19 antibody. The term “PBD-ADC” refers to an ADC in which the drug component is a pyrrolobenzodiazepine (PBD) warhead. The term “anti-CD19-ADC” refers to an ADC in which the antibody component is an anti-CD19 antibody, and the drug component is a PBD warhead.

The ADC may comprise a conjugate of formula L-(DL) p, where DL is of formula I or II:

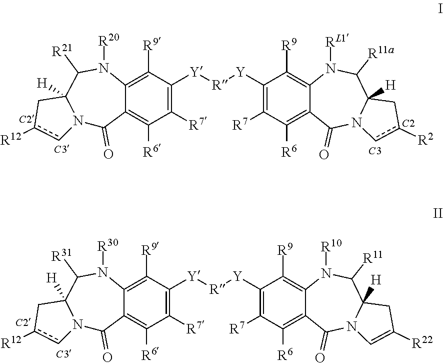

wherein:
L is an antibody (Ab) which is an antibody that binds to CD19;
   when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

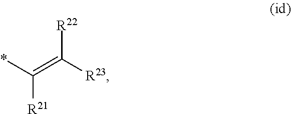

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

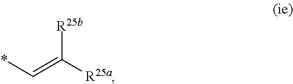

wherein one of R^{25a} and R^{25b} is H and the other is selected from: phenyl, which phenyl is optionally substituted by a group selected from halo, methyl, methoxy; pyridyl; and thiophenyl; and

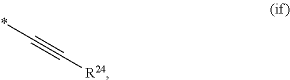

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl; when there is a single bond present between C2′ and C3′,
R¹² is

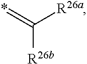

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the antibody (Ab);
R^{11a} is selected from OH, ORA, where RA is C₁₋₄ alkyl, and SO₂M, where z is 2 or 3 and M is a monovalent pharmaceutically acceptable cation;
R²⁰ and R²¹ either together form a double bond between the nitrogen and carbon atoms to which they are bound or;
R²⁰ is selected from H and RC, where RC is a capping group;
R²¹ is selected from OH, ORA and SO₂M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

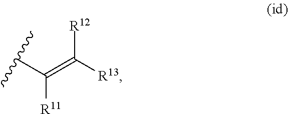

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

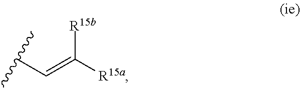

wherein one of R^{15a} and R^{15b} is H and the other is selected from: phenyl, which phenyl is optionally substituted by a group selected from halo, methyl, methoxy; pyridyl; and thiophenyl; and

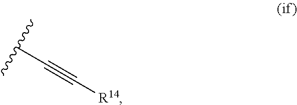

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is

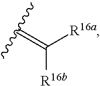

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

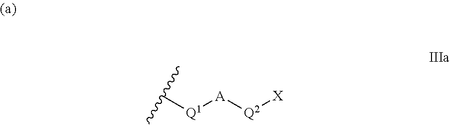

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

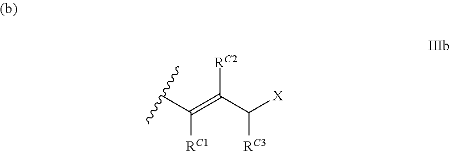

where;
R^{C1}, R^{C2} and R^{C3} are independently selected from H and unsubstituted C₁₋₂ alkyl;

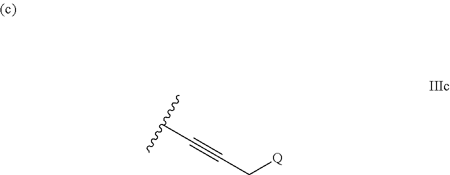

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2′}, NHNH—R^{L2′}, CONHNH—R^{L2′},

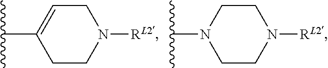

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, ORA and SO₂M;
R³⁰ and R³¹ either together form a double bond between the nitrogen and carbon atoms to which they are bound or;
R³⁰ is H and R³¹ is selected from OH, ORA and SO₂M.

In some embodiments L-R^{L1′} or L-R^{L2′} is a group:

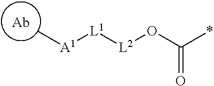

where the asterisk indicates the point of attachment to the PBD, Ab is the antibody, L¹ is a cleavable linker, A is a connecting group connecting L¹ to the antibody, L² is a covalent bond or together with —OC(═O)— forms a self-immolative linker.

In some of these embodiments, L¹ is enzyme cleavable.

In one embodiment, the PBD is of formula (III):

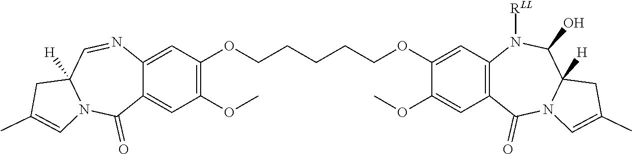

wherein R^{LL} is a linker for connection to Ab.

In another embodiment, the PBD is of formula (IV):

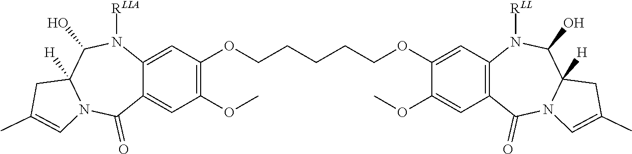

wherein R^{LL} is a linker for connection to Ab and R^{LLA} is a linker for connection to Ab or capping group RC.

It has previously been shown that such ADCs are useful in the treatment of CD19 expressing cancers (see, for example, WO2014/057117, WO2018/193105, and WO2018/229222 which are incorporated by reference herein in their entirety).

The term anti-CD19-ADC may include any embodiment described in WO2014/057117. In particular, in preferred embodiments the ADC may have the chemical structure:

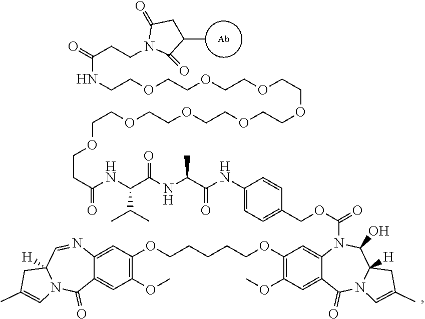

where the Ab is a CD19 antibody, and the DAR is between 1 and 8.

The antibody may comprise a VH domain having the sequence according to any one of SEQ ID NOs. 1, 2, 3, 4, 5 or 6, optionally further comprising a VL domain having the sequence according to any one of SEQ ID NOs. 7, 8, 9, 10, 11 or 12.

In some aspects the antibody component of the anti-CD19-ADC is an antibody comprising: VH and VL domains respectively having the sequences of: SEQ ID NO. 1 and SEQ ID NO. 7, SEQ ID NO. 2 and SEQ ID NO. 8, SEQ ID NO. 3 and SEQ ID NO. 9, SEQ ID NO. 4 and SEQ ID NO. 10, SEQ ID NO. 5 and SEQ ID NO. 11, or SEQ ID NO. 6 and SEQ ID NO. 12.

In some embodiments the antibody has a VH domain comprising a VH CDR1, a VH CDR2, and a VH CDR3, wherein the antibody comprises the CDR sequences of the VH domain having the sequence according to SEQ ID NO: 2. In some embodiments the antibody has a VL domain comprising a VL CDR1, a VL CDR2, and a VL CDR3, wherein the antibody comprises the CDR sequences of the VL domain having the sequence according to SEQ ID NO: 8.

In preferred embodiments the antibody comprises a VH domain and a VL domain,
the VH domain comprising a VH CDR1, a VH CDR2, and a VH CDR3, wherein the antibody comprises the CDR sequences of the VH domain having the sequence according to SEQ ID NO: 2; and
the VL domain comprising a VL CDR1, a VL CDR2, and a VL CDR3, wherein the antibody comprises the CDR sequences of the VL domain having the sequence according to SEQ ID NO: 8.

The CDRs of antibody variable domains described herein may be identified by any suitable method known in the art, for example using any suitable antibody numbering scheme. The CDRs may be identified using any of the Kabat numbering scheme (Kabat et al., U.S. Department of Health and Human Services, 1991), the Chothia numbering scheme (Chothia C, Lesk A M. J Mol Biol. (1987) 196:901-17), or the IMGT numbering scheme (Giudicelli V, et al. Nucleic Acids Res. (1997) 25:206-11; Lefranc M P. Immunol Today (1997) 18:509). The skilled person will appreciate that these different CDR labelling systems can give slightly different results, but in each case the CDRs can be easily identified by the skilled person.

In preferred embodiments the antibody comprises a VH domain having the sequence according to SEQ ID NO. 2. In preferred embodiments the antibody comprises a VL domain having the sequence according to SEQ ID NO. 8.

In preferred embodiments the antibody comprises a VH domain and a VL domain, the VH and domain having the sequence of SEQ ID NO. 2 and the VL domain having the sequences of SEQ ID NO. 8.

The VH and VL domain(s) may pair so as to form an antibody antigen binding site that binds CD19.

In some embodiments the antibody is an intact antibody comprising a VH domain and a VL domain, the VH and VL domains having sequences of SEQ ID NO. 2 and SEQ ID NO. 8.

In some embodiments the antibody is an antibody comprising a heavy chain having sequences of SEQ ID NO. 13 and a light chain having the sequences of SEQ ID NO. 14.

In some embodiments the antibody is a fully human monoclonal IgG1 antibody, preferably IgG1,κ.

In some embodiments the antibody is the RB4v1.2 antibody described in WO2014/057117.

In an aspect the antibody is an antibody as described herein which has been modified (or further modified) as described below. In some embodiments the antibody is a humanised, deimmunised or resurfaced version of an antibody disclosed herein.

The most preferred anti-CD19-ADC for use with the aspects of the present disclosure is ADCx19, as described herein below.

A second preferred anti-CD19-ADC for use with the aspects of the present disclosure is ADCT-402.

### ADCx19

ADCx19 is an antibody drug conjugate composed of a humanized antibody against human CD19 attached to a pyrrolobenzodiazepine (PBD) warhead via a cleavable linker. The mechanism of action of ADCX19 depends on CD19 binding. The CD19 specific antibody targets the antibody drug conjugate (ADC) to cells expressing CD19. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

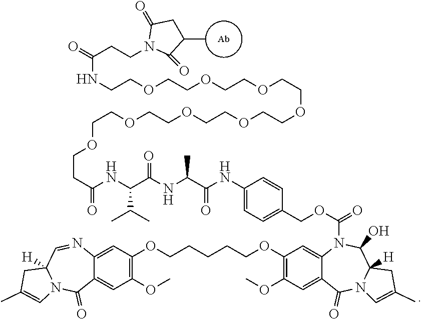

Ab represents Antibody RB4v1.2 (antibody with the VH and VL sequences SEQ ID NO. 2 and SEQ ID NO. 8, respectively). It is synthesised as described in WO2014/057117 (RB4v1.2-E) and typically has a DAR (Drug to Antibody Ratio) of 2+/−0.5, such as +/−0.3.

### CD19 Binding

As used herein, “binds CD19” is used to mean the antibody binds CD19 with a higher affinity than a non-specific partner such as Bovine Serum Albumin (BSA, Genbank accession no. CAA76847, version no. CAA76847.1 GI: 3336842, record update date: Jan. 7, 2011 02:30 PM). In some embodiments the antibody binds CD19 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the invention can bind CD19 with a high affinity. For example, in some embodiments the antibody can bind CD19 with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD19 polypeptide corresponds to Genbank accession no. NP_001171569, version no. NP_001171569.1 GI: 296010921, record update date: Sep. 10, 2012 12:43 AM. In one embodiment, the nucleic acid encoding CD19 polypeptide corresponds to Genbank accession no NM_001178098, version no. NM_001178098.1 GI: 296010920, record update date: Sep. 10, 2012 12:43 AM. In some embodiments, CD19 polypeptide corresponds to Uniprot/Swiss-Prot accession No. P15391.

The present disclosure also relates more generally to a combination of an antibody drug conjugate that comprises a PBD dimer as the warhead (such the PBDs described herein) and an antibody drug conjugate that comprises monomethyl auristatin E as the warhead, and therapeutics methods as described herein which comprise administered a combination of the two antibody drug conjugates. The antibodies for each of the individual agents are selected to bind to a different target molecule.

One example of an antibody drug conjugate that comprises monomethyl auristatin E is where the antibody targets anti-CD79b.

Examples of antibody drug conjugates that comprises a PBD dimer, are those that target CD19 as described above as well as those that target CD25 or CD22, as described below.

It will be appreciated by a person skilled in the art that further embodiments described throughout in detail for anti-CD19 ADCs and anti-CD79b, such as the selection of PBD, disorders to be treated, patient selection and administration, can be applied mutatis mutandis to other ADCs described herein that bind to different targets. Particular examples of other targets are CD25 and CD22 which are described in more detail below.

### Anti-CD25-ADCs

The disclosure also relates to treatment with an anti-CD25 ADC disclosed in WO2014/057119, and as herein described.

As used herein, the terms “anti-CD25 ADC” or “CD25-ADC” refers to an ADC in which the antibody component is an anti-CD25 antibody. The term “PBD-ADC” refers to an ADC in which the drug component is a pyrrolobenzodiazepine (PBD) warhead. The term “anti-CD25-ADC” refers to an ADC in which the antibody component is an anti-CD25 antibody, and the drug component is a PBD warhead. The ADC component is as defined above for anti-CD19-ADCs.

In some embodiments the antibody has a VH domain comprising a VH CDR1, a VH CDR2, and a VH CDR3, wherein the antibody comprises the CDR sequences of the VH domain having the sequence according to SEQ ID NO: 25. In some embodiments the antibody has a VL domain comprising a VL CDR1, a VL CDR2, and a VL CDR3, wherein the antibody comprises the CDR sequences of the VL domain having the sequence according to SEQ ID NO: 25.

In preferred embodiments the antibody comprises a VH domain and a VL domain,
the VH domain comprising a VH CDR1, a VH CDR2, and a VH CDR3, wherein the antibody comprises the CDR sequences of the VH domain having the sequence according to SEQ ID NO: 25; and
the VL domain comprising a VL CDR1, a VL CDR2, and a VL CDR3, wherein the antibody comprises the CDR sequences of the VL domain having the sequence according to SEQ ID NO: 26.

The antibody may comprise a VH domain comprising a VH CDR1 with the amino acid sequence of SEQ ID NO. 27, a VH CDR2 with the amino acid sequence of SEQ ID NO. 28, and a VH CDR3 with the amino acid sequence of SEQ ID NO. 29.

In some aspects the antibody component of the anti-CD25-ADC is an antibody comprising: a VH domain comprising a VH CDR1 with the amino acid sequence of SEQ ID NO. 27, a VH CDR2 with the amino acid sequence of SEQ ID NO. 28, and a VH CDR3 with the amino acid sequence of SEQ ID NO. 29. In some embodiments the antibody comprises a VH domain having the sequence according to SEQ ID NO. 25.

The antibody may further comprise: a VL domain comprising a VL CDR1 with the amino acid sequence of SEQ ID NO. 30, a VL CDR2 with the amino acid sequence of SEQ ID NO. 31, and a VL CDR3 with the amino acid sequence of SEQ ID NO. 32. In some embodiments the antibody further comprises a VL domain having the sequence according to SEQ ID NO. 26.

In some embodiments the antibody comprises a VH domain and a VL domain, the VH and VL domains having the sequences of SEQ ID NO. 25 paired with SEQ ID NO. 26.

The VH and VL domain(s) may pair so as to form an antibody antigen binding site that binds CD25.

In preferred embodiments the antibody is an intact antibody comprising a VH domain and a VL domain, the VH and VL domains having sequences of SEQ ID NO. 25 and SEQ ID NO. 26.

In some embodiments the antibody is a fully human monoclonal IgG1 antibody, preferably IgG1,κ.

In some embodiments the antibody is the AB12 antibody described in WO 2004/045512 (Genmab A/S).

In an aspect the antibody is an antibody as described herein which has been modified (or further modified) as described below. In some embodiments the antibody is a humanised, deimmunised or resurfaced version of an antibody disclosed herein.

A preferred anti-CD25-ADC for use with the aspects of the present disclosure is ADCX25, as described herein below.

Another preferred anti-CD25-ADC for use with the aspects of the present disclosure is camidanlumab tesirine.

### ADCx25

ADCx25 is an antibody drug conjugate composed of a human antibody against human CD25 attached to a pyrrolobenzodiazepine (PBD) warhead via a cleavable linker. The mechanism of action of ADCX25 depends on CD25 binding. The CD25 specific antibody targets the antibody drug conjugate (ADC) to cells expressing CD25. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period (Hartley 2011).

It has the chemical structure:

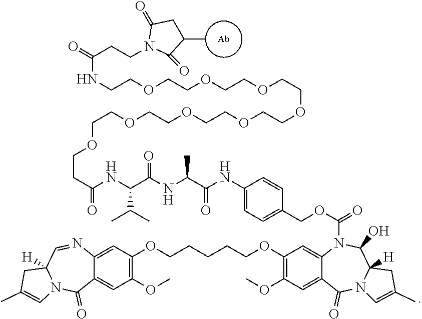

Ab represents Antibody AB12 (fully human monoclonal IgG1, K antibody with the VH and VL sequences SEQ ID NO. 25 and SEQ ID NO. 26, respectively, also known as HuMax-TAC). It is synthesised as described in WO 2014/057119 (Conj AB12-E) and typically has a DAR (Drug to Antibody Ratio) of 2.0+/−0.3.

### CD25 Binding

As used herein, “binds CD25” is used to mean the antibody binds CD25 with a higher affinity than a non-specific partner such as Bovine Serum Albumin (BSA, Genbank accession no. CAA76847, version no. CAA76847.1 GI: 3336842, record update date: Jan. 7, 2011 02:30 PM). In some embodiments the antibody binds CD25 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the disclosure can bind CD25 with a high affinity. For example, in some embodiments the antibody can bind CD25 with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD25 polypeptide corresponds to Genbank accession no. NP_000408, version no. NP_000408.1 GI: 4557667, record update date: Sep. 9, 2012 04:59 PM. In one embodiment, the nucleic acid encoding CD25 polypeptide corresponds to Genbank accession no. NM_000417, version no. NM_000417.2 GI: 269973860, record update date: Sep. 9, 2012 04:59 PM. In some embodiments, CD25 polypeptide corresponds to Uniprot/Swiss-Prot accession No. P01589.

### Anti-CD22-ADCs

The disclosure also relates to treatment with an anti-CD22 ADC disclosed in WO2014/057122, and as herein described.

As used herein, the terms “anti-CD22 ADC” or “CD22-ADC” refers to an ADC in which the antibody component is an anti-CD22 antibody. The term “PBD-ADC” refers to an ADC in which the drug component is a pyrrolobenzodiazepine (PBD) warhead. The term “anti-CD22-ADC” refers to an ADC in which the antibody component is an anti-CD22 antibody, and the drug component is a PBD warhead. The ADC component is as defined above for anti-CD19-ADCs.

### the Antibody Component of the Anti-CD22 ADC

The antibody may comprise an amino acid substitution of an interchain cysteine residue by an amino acid that is not cysteine, wherein the conjugation of the drug moiety to the antibody is at an interchain cysteine residue

The antibody preferably comprises: (i) a heavy chain having an amino acid substitution of each of the interchain cysteine residues HC226 and HC229 according to the EU index as set forth in Kabat; (ii) a light chain having an amino acid substitution of the interchain cysteine residue KLC214 or ALC213 according to the EU index as set forth in Kabat; and (iii) a heavy chain retaining the unsubstituted interchain cysteine HC220 according to the EU index as set forth in Kabat.

Preferably the drug moiety is conjugated to the unsubstituted interchain cysteine HC220. The interchain cysteine residues HC226 and HC229 may each be substituted for valine. The interchain cysteine residues KLC214 or ALC213 may be substituted for serine.

In some embodiments, the antibody of the conjugates described herein comprises a light chain comprising the amino acid sequence of SEQ ID NO. 45, or fragment thereof, wherein the cysteine at position 105, if present, is substituted by an amino acid that is not cysteine. For example, SEQ ID NO. 46 discloses a light chain comprising the amino acid sequence of SEQ ID NO. 45 wherein the cysteine at position 105 is substituted by a serine residue.

In some embodiments, the antibody of the conjugates described herein comprises a light chain comprising the amino acid sequence of SEQ ID NO. 47, or fragment thereof, wherein the cysteine at position 102, if present, is substituted by an amino acid that is not cysteine. For example, SEQ ID NO. 48 discloses a light chain comprising the amino acid sequence of SEQ ID NO. 47 wherein the cysteine at position 102 is substituted by a serine residue.

In some embodiments the antibody comprises:
(i) a heavy chain having an amino acid substitution of each of the interchain cysteine residues HC226 and HC229 according to the EU index as set forth in Kabat, optionally wherein HC226 and HC229 is each substituted for valine;
(ii) a light chain having an amino acid substitution of the interchain cysteine residue KLC214 or ALC213 according to the EU index as set forth in Kabat, optionally wherein KLC214 or ALC213 is substituted for serine;
(iii) a heavy chain retaining the unsubstituted interchain cysteine HC220 according to the EU index as set forth in Kabat, optionally wherein the drug moiety is conjugated to the cysteine at HC220. In these embodiments, the antibody preferably further comprises a VH domain and VL domain as defined herein below. The light chain may comprise the amino acid sequence of: (i) SEQ ID NO. 45, or fragment thereof, wherein the cysteine at position 105, if present, is substituted by an amino acid that is not cysteine (such as in SEQ ID NO. 46); or SEQ ID NO. 47, or fragment thereof, wherein the cysteine at position 102, if present, is substituted by an amino acid that is not cysteine (such as in SEQ ID NO. 48).

The antibody may comprise a heavy chain comprising the amino acid sequence of SEQ ID NO. 43, and a light chain comprising the amino acid sequence of SEQ ID NO. 45 or SEQ ID NO. 47;
wherein each of the cysteines at positions 109 and 112 in SEQ ID NO: 43 is substituted by an amino acid that is not cysteine;
and wherein the cysteine at position 105 in SEQ ID NO: 45 or the cysteine at position 102 in SEQ ID NO: 47, is substituted by an amino acid that is not cysteine. Preferably the drug moiety is conjugated to the cysteine at position 103 of SEQ ID NO. 43. In some embodiments the cysteines at positions 109 and 112 in SEQ ID NO: 43 are substituted for valine, such as in SEQ ID NO: 44. In some embodiments the cysteine at position 105 in SEQ ID NO: 45 or the cysteine at position 102 in SEQ ID NO: 47 is substituted by serine such as in SEQ ID NOs: 46 and 48.

In some embodiments the antibody is a fully human monoclonal IgG1 antibody, preferably IgG1,κ.

### the VH and VL Domains

In some embodiments the antibody has a VH domain comprising a VH CDR1, a VH CDR2, and a VH CDR3, wherein the antibody comprises the CDR sequences of the VH domain having the sequence according to SEQ ID NO: 1. In some embodiments the antibody has a VL domain comprising a VL CDR1, a VL CDR2, and a VL CDR3, wherein the antibody comprises the CDR sequences of the VL domain having the sequence according to SEQ ID NO: 34.

In preferred embodiments the antibody comprises a VH domain and a VL domain,
the VH domain comprising a VH CDR1, a VH CDR2, and a VH CDR3, wherein the antibody comprises the CDR sequences of the VH domain having the sequence according to SEQ ID NO: 33; and
the VL domain comprising a VL CDR1, a VL CDR2, and a VL CDR3, wherein the antibody comprises the CDR sequences of the VL domain having the sequence according to SEQ ID NO: 34.

The antibody may comprise a VH domain comprising a VH CDR1 with the amino acid sequence of SEQ ID NO. 35, a VH CDR2 with the amino acid sequence of SEQ ID NO. 36, and a VH CDR3 with the amino acid sequence of SEQ ID NO. 37.

In some aspects the antibody component of the anti-CD22-ADC is an antibody comprising: a VH domain comprising a VH CDR1 with the amino acid sequence of SEQ ID NO. 35, a VH CDR2 with the amino acid sequence of SEQ ID NO. 36, and a VH CDR3 with the amino acid sequence of SEQ ID NO. 37. In some embodiments the antibody comprises a VH domain having the sequence according to SEQ ID NO. 22.

The antibody may further comprise: a VL domain comprising a VL CDR1 with the amino acid sequence of SEQ ID NO. 38, a VL CDR2 with the amino acid sequence of SEQ ID NO. 39, and a VL CDR3 with the amino acid sequence of SEQ ID NO. 40. In some embodiments the antibody further comprises a VL domain having the sequence according to SEQ ID NO. 34.

In some embodiments the antibody comprises a VH domain and a VL domain, the VH and VL domains having the sequences of SEQ ID NO. 33 paired with SEQ ID NO. 34.

The VH and VL domain(s) may pair so as to form an antibody antigen binding site that binds CD22.

In some aspects the antibody component of the anti-CD22-ADC is an antibody comprising: a VH domain having the sequence according to SEQ ID NO. 33.

The antibody may further comprise a VL domain having the sequence according to SEQ ID NO. 34.

In some embodiments the antibody comprises a VH domain and VL domain as described herein below. In some embodiments, the antibody comprises:
a heavy chain having the sequence according to SEQ ID NO: 44;
a light chain having the sequence according to SEQ ID NO: 46;
a VH domain having the sequence according to SEQ ID NO. 33; and
a VL domain having the sequence according to SEQ ID NO. 32.

Preferably the drug moiety is conjugated to the cysteine at position 103 of SEQ ID NO. 44.

In some embodiments the antibody is the epratuzumab antibody described in WO2014/057122.

In come embodiments the antibody comprises a heavy chain having the sequence according to SEQ ID NO. 47 and a light chain having the sequence according to SEQ ID NO. 48. Preferably the drug moiety is conjugated to the cysteine at position 219 of SEQ ID NO. 47.

In an aspect the antibody is an antibody as described herein which has been modified (or further modified) as described below. In some embodiments the antibody is a humanised, deimmunised or resurfaced version of an antibody disclosed herein.

The most preferred anti-CD22-ADC for use with the aspects of the present disclosure is ADCx22, as described herein below.

### ADCx22

ADCx22 is an antibody drug conjugate composed of a human antibody against human CD22 attached to a pyrrolobenzodiazepine (PBD) warhead via a cleavable linker. The mechanism of action of ADCX22 depends on CD22 binding. The CD22 specific antibody targets the antibody drug conjugate (ADC) to cells expressing CD22. Upon binding, the ADC internalizes and is transported to the lysosome, where the protease sensitive linker is cleaved and free PBD dimer is released inside the target cell. The released PBD dimer inhibits transcription in a sequence-selective manner, due either to direct inhibition of RNA polymerase or inhibition of the interaction of associated transcription factors. The PBD dimer produces covalent crosslinks that do not distort the DNA double helix and which are not recognized by nucleotide excision repair factors, allowing for a longer effective period.

It has the chemical structure:

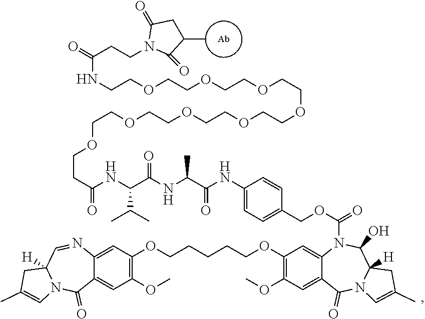

wherein Ab represents an antibody comprising a VH domain having the sequence of SEQ ID NO. 33 and a VL domain having the sequence of SEQ ID NO. 34. Typically the antibody further comprises: (i) a heavy chain having an amino acid substitution of each of the interchain cysteine residues HC226 and HC229 according to the EU index as set forth in Kabat (for example, to valine); (ii) a light chain having an amino acid substitution of the interchain cysteine residue KLC214 according to the EU index as set forth in Kabat (for example, to serine); and (iii) a heavy chain retaining the unsubstituted interchain cysteine HC220 according to the EU index as set forth in Kabat. Typically the drug moiety is conjugated to the cysteine at HC220.

Accordingly, the antibody typically comprises a heavy chain having the sequence according to SEQ ID NO. 41 and a light chain having the sequence according to SEQ ID NO. 42. Linkage to the drug occurs on Heavy Chain interchain cysteine Cys220 (EU numbering). HC220 corresponds to position 219 of SEQ ID NO. 41.

It is noted that “having the sequence” has the same meaning as “comprising the sequence”; in particular, in some embodiments the heavy chain of ADCx22 is expressed with an additional terminal ‘K’ residue (so, ending . . . SPGK), with the terminal K being optionally removed post-translationally to improve the homogeneity of the final therapeutic ADC product.

### CD22 Binding

As used herein, “binds CD22” is used to mean the antibody binds CD22 with a higher affinity than a non-specific partner such as Bovine Serum Albumin (BSA, Genbank accession no. CAA76847, version no. CAA76847.1 GI: 3336842, record update date: Jan. 7, 2011 02:30 PM). In some embodiments the antibody binds CD22 with an association constant (Kₐ) at least 2, 3, 4, 5, 10, 20, 50, 100, 200, 500, 1000, 2000, 5000, 10⁴, 10⁵ or 10⁶-fold higher than the antibody's association constant for BSA, when measured at physiological conditions. The antibodies of the invention can bind CD22 with a high affinity. For example, in some embodiments the antibody can bind CD22 with K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

In some embodiments, CD22 polypeptide corresponds to Genbank accession no. BAB15489, version no. BAB15489.1 GI: 10439338, record update date: Sep. 11, 2006 11:24 PM. In one embodiment, the nucleic acid encoding CD22 polypeptide corresponds to Genbank accession no AK026467, version no. AK026467.1 GI: 10439337, record update date: Sep. 11, 2006 11:24 PM.

### Anti-CD79b Agents

### CD79b

CD79 (composed of subunits CD79a and CD79b) is a heterodimeric signal-transduction component of the B-cell receptor. CD79b membrane expression is restricted to the B-cell compartment and is ubiquitously expressed in mature B-cell lymphomas and placed on the cell surface by the earliest committed B-cell progenitors before expression of immunoglobulin u. Antibodies to CD79b induce negative cell signals and suppress response to T-cell-dependent antigens (Nakamura et al., Int J Hematol. 1996; 64:39-46). However, unconjugated anti-CD79b antibodies induce modest B-cell depletion and show moderate antibody-dependent and complement-dependent cellular cytotoxicity, if any (Fuh et al., Br J Pharmacol. 2017; 174:628-640). Conversely, anti-CD79b ADCs are trafficked to a lysosomal-like compartment of B cells as part of antigen presentation, (Polson et al., Blood. 2007; 110:616-623) and induce a prolonged and sustained depletion of proliferating B cells (Fuh et al., ibid.).

### Anti-CD79b Agents

‘Anti-CD79b agent’ is used herein to mean any agent that specifically binds to CD79b and/or a cell that expresses CD79b. Preferably the agent induces B-cell depletion.

As used herein, “specifically binds CD79b” is used to mean the agent binds CD79b with a higher affinity than a non-specific partner such as Bovine Serum Albumin (BSA, Genbank accession no. CAA76847, version no. CAA76847.1 GI: 3336842, record update date: Jan. 7, 2011 02:30 PM). In some embodiments the agent binds CD79b with an association constant (Kₐ) at least 2, 3, 4, 5, 10, 20, 50, 100, 200, 500, 1000, 2000, 5000, 10⁴, 10⁵ or 10⁶-fold higher than the agent's association constant for BSA, when measured at physiological conditions. The agents may bind CD79b with a high affinity. For example, in some embodiments the agent can bind CD79b with a K_{D} equal to or less than about 10⁻⁶ M, such as 1×10⁻⁶, 10⁻⁷, 10⁻⁸, 10⁻⁹, 10⁻¹⁰, 10⁻¹¹, 10⁻¹², 10⁻¹³ or 10⁻¹⁴.

### Anti-CD79b ADCs

Anti-CD79b ADC is used herein to mean a conjugate comprising a moiety that specifically binds to CD79b conjugated to a payload.

In some embodiments the moiety that specifically binds to CD79b is an antibody. In some cases the antibody is Polatuzumab.

In some embodiments the payload comprises a drug, such as a cytotoxic drug. In some cases the cytotoxic drug is an auristatin, such as a monomethyl auristatin. In some cases the cytotoxic drug is monomethyl auristatin (MMAE).

In some cases the payload comprises a linker moiety through which the payload I conjugated to the moiety that specifically binds to CD79b. In some embodiments the payload is a drug-linker, wherein the drug-linker comprises a drug moiety and a linker moiety. The linker moiety may be cleavable by an enzyme such as a protease. For example, in some embodiments the linker is a dipeptide such as Valine-Citrulline (val-cit, or vc).

In some embodiments the ADC has the structure:

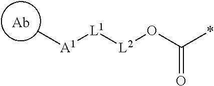

wherein the asterisk indicates the point of attachment to the drug moiety, Ab is the antibody, L¹ is a cleavable linker, A is a connecting group connecting L¹ to the antibody, L² is a covalent bond or together with —OC(═O)— forms a self-immolative linker. In some of these embodiments, L¹ is enzyme cleavable.

In preferred embodiments the ADC may be a conjugate of formula (I):

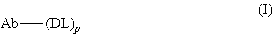

wherein:
Ab is an antibody that binds to CD79b;
DL is

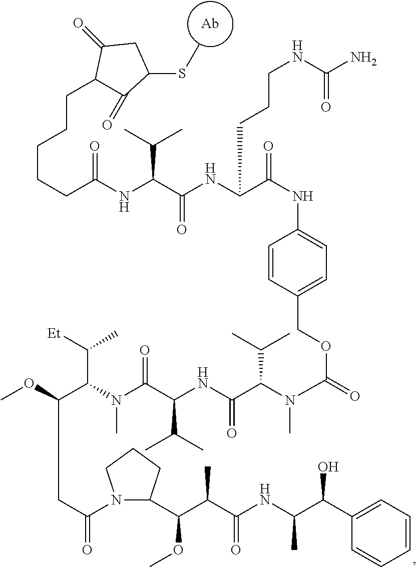 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCC2C(=O)CC(SC)C2=O)C(C)C)cc1)C(C)C]

and p is between 1 and 8, such as between 3 and 4, for example about 3.5.

In some embodiments the antibody has a VH domain comprising a VH CDR1, a VH CDR2, and a VH CDR3, wherein the antibody comprises the CDR sequences of the VH domain having the sequence according to SEQ ID NO: 17. In some embodiments the antibody has a VL domain comprising a VL CDR1, a VL CDR2, and a VL CDR3, wherein the antibody comprises the CDR sequences of the VL domain having the sequence according to SEQ ID NO: 18.

In preferred embodiments the antibody comprises a VH domain and a VL domain,
the VH domain comprising a VH CDR1, a VH CDR2, and a VH CDR3, wherein the antibody comprises the CDR sequences of the VH domain having the sequence according to SEQ ID NO: 17; and
the VL domain comprising a VL CDR1, a VL CDR2, and a VL CDR3, wherein the antibody comprises the CDR sequences of the VL domain having the sequence according to SEQ ID NO: 18.

In some embodiments the antibody comprises a VH domain having a VH CDR3 with the amino acid sequence of SEQ ID NO. 21. In some embodiments the VH domain further comprises a VH CDR2 with the amino acid sequence of SEQ ID NO. 20, and/or a VH CDR1 with the amino acid sequence of SEQ ID NO. 19. In some embodiments the antibody comprises a VH domain having a VH CDR1 with the amino acid sequence of SEQ ID NO. 19, a VH CDR2 with the amino acid sequence of SEQ ID NO. 20, and a VH CDR3 with the amino acid sequence of SEQ ID NO. 21. In some embodiments the antibody a VH domain comprising a VH CDR1, a VH CDR2, and a VH CDR3, wherein the antibody comprises the CDR sequences of the VH domain having the sequence of SEQ ID NO: 17. In preferred embodiments the antibody comprises a VH domain having the sequence according to SEQ ID NO. 17.

The antibody may further comprise a VL domain. In some embodiments the antibody comprises a VL domain having a VL CDR3 with the amino acid sequence of SEQ ID NO. 24. In some embodiments the VL domain further comprises a VL CDR2 with the amino acid sequence of SEQ ID NO. 23, and/or a VL CDR1 with the amino acid sequence of SEQ ID NO. 22. In some embodiments the antibody comprises a VL domain having a VL CDR1 with the amino acid sequence of SEQ ID NO. 22, a VL CDR2 with the amino acid sequence of SEQ ID NO. 23, and a VL CDR3 with the amino acid sequence of SEQ ID NO. 24. In some embodiments the antibody a VL domain comprising a VL CDR1, a VL CDR2, and a VL CDR3, wherein the antibody comprises the CDR sequences of the VL domain having the sequence of SEQ ID NO: 18. In preferred embodiments the antibody comprises a VL domain having the sequence according to SEQ ID NO. 18.

The CDRs of antibody variable domains described herein may be identified by any suitable method known in the art, for example using any suitable antibody numbering scheme. The CDRs may be identified using any of the Kabat numbering scheme (Kabat et al., U.S. Department of Health and Human Services, 1991), the Chothia numbering scheme (Chothia C, Lesk A M. J Mol Biol. (1987) 196:901-17), or the IMGT numbering scheme (Giudicelli V, et al. Nucleic Acids Res. (1997) 25:206-11; Lefranc M P. Immunol Today (1997) 18:509). The skilled person will appreciate that these different CDR labelling systems can give slightly different results, but in each case the CDRs can be easily identified by the skilled person.

The VH and VL domain(s) may form an antibody antigen binding site that binds CD79b.

In some embodiments the antibody is an intact antibody comprising a VH domain and a VL domain, the VH and VL domains having sequences of SEQ ID NO. 17 paired with SEQ ID NO. 18.

In some embodiments the antibody is a fully human monoclonal IgG1 antibody, preferably IgG1,κ.

In an aspect the antibody is an antibody as described herein which has been modified (or further modified) as described below. In some embodiments the antibody is a humanised, deimmunised or resurfaced version of an antibody disclosed herein.

The most preferred anti-CD79b agent is Polatuzumab vedotin.

### Polatuzumab Vedotin

Polatuzumab vedotin (Polivy, Roche) is a CD79b-directed antibody-drug conjugate comprised of a humanized IgG1 anti-CD79b mAb conjugated to monomethyl auristatin E (MMAE) via the protease-cleavable linker vc (Val-Cit).

It has the chemical structure:

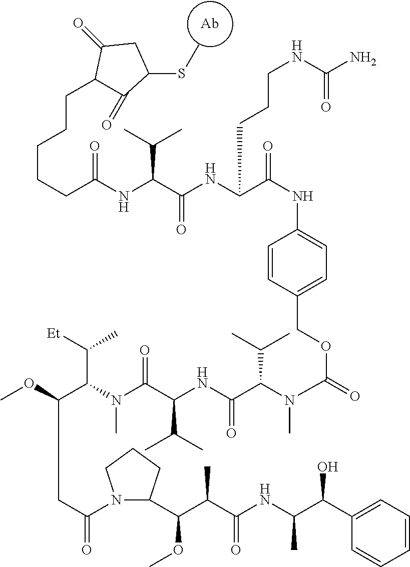 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCC2C(=O)CC(SC)C2=O)C(C)C)cc1)C(C)C]

Ab represents antibody Polatuzumab (antibody with the VH and VL sequences SEQ ID NO. 17 and SEQ ID NO. 18, respectively). It typically has a DAR (Drug to Antibody Ratio) of 3.5.

### Advantageous Properties of the Described Combinations

Both the anti-CD19 ADC (or anti-CD25 ADC or anti-CD22 ADC) and anti-CD79b agent when used as a single agent in isolation have demonstrated clinical utility—for example, in the treatment of cancer. However, as described herein, combination of the anti-CD19 ADC and anti-CD79b agent is expected to provide one or more of the following advantages over treatment with either anti-CD19 ADC or anti-CD79b agent alone:
1) effective treatment of a broader range of cancers;
2) effective treatment of resistant or refractory forms of disorders such as cancer, and individuals with disorders such as cancer who have relapsed after a period of remission;
3) increased response rate to treatment; and/or
4) Increased durability of treatment.

Effective treatment of a broader range of cancers as used herein means that following treatment with the combination a complete response is observed with a greater range of recognised cancer types. That is, a complete response is seen from cancer types not previously reported to completely respond to either anti-CD19 ADC or anti-CD79b agent alone.

Without wishing to be bound by theory, in embodiments where the anti-CD19 ADC and anti-CD79b agent comprise different classes of cytotoxic drug (for example where the anti-CD19 ADC is Loncastuximab tesirine and the anti-CD79b agent is Pola-V) that have different mode of actions (PBD dimers are DNA cross-linking agents while MMAE are tubulin inhibitor), the cellular toxicity acting through two distinct pathways is believed to contribute to the additive or synergistic cytotoxicity. Moreover, the fact the two agents target two different cell surface antigens means that they are not competing for binding to the same antigen on the cell surface. This facilitates delivery of the cytotoxic drugs into the target cell.

Effective treatment of a resistant, refractory, or relapsed forms as used herein means that following treatment with the combination a complete response is observed in individuals that are either partially or completely resistant or refractory to treatment with either anti-CD19 ADC or anti-CD79b agent alone (for example, individuals who show no response or only partial response following treatment with either agent alone, or those with relapsed disorder). In some embodiments, a complete response following treatment with the anti-CD19 ADC/anti-CD79b agent combination is observed at least 10% of individuals that are either partially or completely resistant or refractory to treatment with either anti-CD19 ADC or anti-CD79b agent alone. In some embodiments, a complete response following treatment with the anti-CD19 ADC/anti-CD79b agent combination is observed at least 20%, at least 30%, at least 40%, at least 50%, at least 60%, at least 70%, at least 80%, at least 90%, at least 95%, at least 98%, or at least 99% of individuals that are either partially or completely resistant or refractory to treatment with either anti-CD19 ADC or anti-CD79b agent alone.

Increased response rate to treatment as used herein means that following treatment with the combination a complete response is observed in a greater proportion of individuals than is observed following treatment with either anti-CD19 ADC or anti-CD79b agent alone. In some embodiments, a complete response following treatment with the anti-CD19 ADC/anti-CD79b agent combination is observed at least 10% of treated individuals. In some embodiments, a complete response following treatment with the anti-CD19 ADC/anti-CD79b agent combination is observed at least 20%, at least 30%, at least 40%, at least 50%, at least 60%, at least 70%, at least 80%, at least 90%, at least 95%, at least 98%, or at least 99% of treated individuals.

Increased durability of treatment as used herein means that average duration of complete response in individuals treated with the combination is longer than in individuals who achieve complete response following treatment with either anti-CD19 ADC or anti-CD79b agent alone. In some embodiments, the average duration of a complete response following treatment with the anti-CD19 ADC/anti-CD79b agent combination is at least 6 months. In some embodiments, the average duration of a complete response following treatment with the anti-CD19 ADC/anti-CD79b agent combination is at least 12 months, at least 18 months, at least 24 months, at least 3 years, at least 4 years, at least 5 years, at least 6 years, at least 7 years, at least 8 years, at least 9 years, at least 10 years, at least 15 years, or at least 20 years.

‘Complete response’ is used herein to mean the absence of any clinical evidence of disease in an individual. Evidence may be assessed using the appropriate methodology in the art, for example CT or PET scanning, or biopsy where appropriate. The number of doses required to achieve complete response may be one, two, three, four, five, ten or more. In some embodiments the individuals achieve complete response no more than a year after administration of the first dose, such as no more than 6 months, no more than 3 months, no more than a month, no more than a fortnight, or no more than a week after administration of the first dose.

References to anti-CD19 in this section and in all the following sections can in other embodiments be replaced with anti-CD22 and anti-CD25, unless specifically indicated, mutatis mutandis.

### Treated Disorders

The combined therapies described herein include those with utility for anticancer activity. In particular, in certain aspects the therapies include an antibody conjugated, i.e. covalently attached by a linker, to a PBD drug moiety, i.e. toxin. When the drug is not conjugated to an antibody, the PBD drug has a cytotoxic effect. The biological activity of the PBD drug moiety is thus modulated by conjugation to an antibody. The antibody-drug conjugates (ADC) of the disclosure selectively deliver an effective dose of a cytotoxic agent to tumor tissue whereby greater selectivity, i.e. a lower efficacious dose, may be achieved.

Thus, in one aspect, the present disclosure provides combined therapies comprising administering an anti-CD19 ADC which binds CD19 for use in therapy, wherein the method comprises selecting a subject based on expression of the target protein.

In one aspect, the present disclosure provides a combined therapy with a label that specifies that the therapy is suitable for use with a subject determined to be suitable for such use. The label may specify that the therapy is suitable for use in a subject has expression of CD19, such as overexpression of CD19. The label may specify that the subject has a particular type of cancer.

The cancer may be lymphoma, such as non-Hodgkins lymphoma. The label may specify that the subject has a CD19+ lymphoma.

In a further aspect there is also provided a combined therapy as described herein for use in the treatment of a proliferative disease. Another aspect of the present disclosure provides the use of a conjugate compound in the manufacture of a medicament for treating a proliferative disease.

One of ordinary skill in the art is readily able to determine whether or not a candidate combined therapy treats a proliferative condition for any particular cell type. For example, assays which may conveniently be used to assess the activity offered by a particular compound are described below.

The combined therapies described herein may be used to treat a proliferative disease. The term “proliferative disease” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

Proliferative disorders of particular interest include, but are not limited to, non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL). [Fielding A., Haematologica. 2010 January; 95 (1): 8-12].

It is contemplated that the combined therapies of the present disclosure may be used to treat various diseases or disorders, e.g. characterized by the overexpression of a tumor antigen. Exemplary conditions or hyperproliferative disorders include benign or malignant tumors; leukemia, haematological, and lymphoid malignancies. Others include neuronal, glial, astrocytal, hypothalamic, glandular, macrophagal, epithelial, stromal, blastocoelic, inflammatory, angiogenic and immunologic, including autoimmune disorders and graft-versus-host disease (GVHD).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Autoimmune diseases for which the combined therapies may be used in treatment include rheumatologic disorders (such as, for example, rheumatoid arthritis, Sjögren's syndrome, scleroderma, lupus such as SLE and lupus nephritis, polymyositis/dermatomyositis, cryoglobulinemia, anti-phospholipid antibody syndrome, and psoriatic arthritis), osteoarthritis, autoimmune gastrointestinal and liver disorders (such as, for example, inflammatory bowel diseases (e.g. ulcerative colitis and Crohn's disease), autoimmune gastritis and pernicious anemia, autoimmune hepatitis, primary biliary cirrhosis, primary sclerosing cholangitis, and celiac disease), vasculitis (such as, for example, ANCA-associated vasculitis, including Churg-Strauss vasculitis, Wegener's granulomatosis, and polyarteriitis), autoimmune neurological disorders (such as, for example, multiple sclerosis, opsoclonus myoclonus syndrome, myasthenia gravis, neuromyelitis optica, Parkinson's disease, Alzheimer's disease, and autoimmune polyneuropathies), renal disorders (such as, for example, glomerulonephritis, Goodpasture's syndrome, and Berger's disease), autoimmune dermatologic disorders (such as, for example, psoriasis, urticaria, hives, pemphigus vulgaris, bullous pemphigoid, and cutaneous lupus erythematosus), hematologic disorders (such as, for example, thrombocytopeniarpura, thrombotic thrombocytopenia purpura, post-transfusion purpura, and autoimmune hemolytic anemia), atherosclerosis, uveitis, autoimmune hearing diseases (such as, for example, inner ear disease and hearing loss), Behcet's disease, Raynaud's syndrome, organ transplant, graft-versus-host disease (GVHD), and autoimmune endocrine disorders (such as, for example, diabetic-related autoimmune diseases such as insulin-dependent diabetes mellitus (IDDM), Addison's disease, and autoimmune thyroid disease (e.g. Graves' disease and thyroiditis)). More preferred such diseases include, for example, rheumatoid arthritis, ulcerative colitis, ANCA-associated vasculitis, lupus, multiple sclerosis, Sjögren's syndrome, Graves' disease, IDDM, pernicious anemia, thyroiditis, and glomerulonephritis.

In some aspects, the subject has a proliferative disorder selected from non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

### CD25

The proliferative disease may be characterised by the presence of a neoplasm comprising both CD25+ve and CD25−ve cells. The proliferative disease may be characterised by the presence of a neoplasm composed of CD25−ve neoplastic cells, optionally wherein the CD25−ve neoplastic cells are associated with CD25+ve non-neoplastic cells such as CD25+ve T-cells. The target neoplasm or neoplastic cells may be all or part of a solid tumour.

“Solid tumor” herein will be understood to include solid haematological cancers such as lymphomas (Hodgkin's lymphoma or non-Hodgkin's B- and T-cell lymphoma) which are discussed in more detail herein.

Solid tumors may be neoplasms, including non-haematological cancers, comprising or composed of CD25+ve neoplastic cells. Solid tumors may be neoplasms, including non-haematological cancers, infiltrated with CD25+ve cells, such as CD25+ve T-cells; such solid tumours may lack expression of CD25 (that is, comprise or be composed of CD25−ve neoplastic cells).

For example, the solid tumour may be a tumour with high levels of infiltrating T-cells, such as infiltrating regulatory T-cells (Treg; Ménétrier-Caux, C., et al., Targ Oncol (2012) 7:15-28; Arce Vargas et al., 2017, Immunity 46, 1-10; Tanaka, A., et al., Cell Res. 2017 January; 27 (1): 109-118). Accordingly, the solid tumour may be pancreatic cancer, breast cancer, colorectal cancer, gastric and oesophageal cancer, leukemia and lymphoma, melanoma, non-small cell lung cancer, ovarian cancer, hepatocellular carcinoma, renal cell carcinoma, and head and neck cancer.

### Patient Selection

In certain aspects, the individuals are selected as suitable for treatment with the combined treatments before the treatments are administered.

As used herein, individuals who are considered suitable for treatment are those individuals who are expected to benefit from, or respond to, the treatment. Individuals may have, or be suspected of having, or be at risk of having cancer. Individuals may have received a diagnosis of cancer. In particular, individuals may have, or be suspected of having, or be at risk of having, lymphoma. In some cases, individuals may have, or be suspected of having, or be at risk of having, a solid cancer that has tumour associated non-tumor cells that express CD19, such as infiltrating cells that express CD19.

In some aspects, individuals are selected on the basis of the amount or pattern of expression of CD19. In some aspects, the selection is based on expression of CD19 at the cell surface.

In certain aspects, the target is a CD79b. In some aspects, the selection is based on expression of a CD79b.

In some aspects, the selection is based on levels of both CD19 and CD79b at the cell surface.

In some cases, expression of the target in a particular tissue of interest is determined. For example, in a sample of lymphoid tissue or tumor tissue. In some cases, systemic expression of the target is determined. For example, in a sample of circulating fluid such as blood, plasma, serum or lymph.

In some aspects, the individual is selected as suitable for treatment due to the presence of target expression in a sample. In those cases, individuals without target expression may be considered not suitable for treatment.

In other aspects, the level of target expression is used to select a individual as suitable for treatment. Where the level of expression of the target is above a threshold level, the individual is determined to be suitable for treatment.

In some aspects, the presence of CD19 and/or in cells in the sample indicates that the individual is suitable for treatment with a combination comprising an anti-CD19 ADC and an anti-CD79b agent. In other aspects, the amount of CD19 and/or expression must be above a threshold level to indicate that the individual is suitable for treatment. In some aspects, the observation that CD19 and/or localisation is altered in the sample as compared to a control indicates that the individual is suitable for treatment.

In some aspects, an individual is indicated as suitable for treatment if cells obtained from lymph node or extra nodal sites react with antibodies against CD19 and/or as determined by IHC.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express CD19. In some aspects disclosed herein, a patient is determined to be suitable for treatment if at least at least 10% of the cells in the sample express CD19.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express. In some aspects disclosed herein, a patient is determined to be suitable for treatment if at least at least 10% of the cells in the sample express.

In some aspects, the individual is selected as suitable for treatment based on their current or previous treatment regime. In some embodiments the individual is selected for treatment with the anti-CD19 ADC if the individual has been treated with an anti-CD79b agent. In some embodiments the individual is selected for treatment with the anti-CD19 ADC if the individual is being treated with an anti-CD79b agent. In some cases the individual is selected for treatment if they are refractory to treatment (or further treatment) with the anti-CD79b agent. In some cases the anti-CD79b agent may be Polatuzumab vedotin. In embodiments where the individual is undergoing, or has undergone, treatment with an anti-CD79b agent, the anti-CD19 ADC may be administered in combination with an anti-CD79b agent, or without continued administration of the anti-CD79b agent.

In some embodiments the anti-CD19 ADC is administered to the selected individual in combination with an anti-CD79b agent. In some embodiments the anti-CD19 ADC is administered to the selected individual without continued administration of an anti-CD79b agent. The anti-CD79b agent is preferably Polatuzumab vedotin.

The term ‘refractory to treatment (or further treatment) with the anti-CD79b agent’ is used herein to mean that the disorder (such as cancer) does not respond, or has ceased to respond, to administration of the anti-CD79b agent when administered as a monotherapy. In some embodiments, individuals with refractory NHL are identified using the response criteria disclosed in Cheson at al., 2014 (South Asian J Cancer. 2014 January-March; 3 (1): 66-70). In that document, non-responders are defined as individuals where there is either (i) a >50% increase from nadir in the sum product of diameters of any previously identified abnormal node, or (ii) an appearance of any new lesion during or at the end of therapy. In some embodiments, individuals with refractory leukaemia are identified as individuals with either stable or progressive disease who have completed one complete treatment cycle, or individual achieving partial response after two or more complete treatment cycles.

### CD25

In some aspects, subjects are selected on the basis they have a neoplasm comprising both CD25+ve and CD25−ve cells. The neoplasm may be composed of CD25−ve neoplastic cells, optionally wherein the CD25−ve neoplastic cells are associated with CD25+ve non-neoplastic cells such as CD25+ve Tregs. The neoplasm or neoplastic cells may be all or part of a solid tumour. The solid tumour may be partially or wholly CD25−ve, and may be infiltrated with CD25+ve cells, such as CD25+ve Tregs. In preferred aspects, the solid tumour is associated with high-levels of CD25+ve infiltrating cells, such as Treg cells. In some aspects, the solid tumour is associated with low-levels of CD25+ve infiltrating cells, such as Treg cells. In some aspects, the solid tumour is not associated with CD25+ve infiltrating cells, such as Treg cells; for example, the levels of CD25+ve cells may be below the detection limit.

### Samples

The sample may comprise or may be derived from: a quantity of blood; a quantity of serum derived from the individual's blood which may comprise the fluid portion of the blood obtained after removal of the fibrin clot and blood cells; a quantity of pancreatic juice; a tissue sample or biopsy; or cells isolated from said individual.

A sample may be taken from any tissue or bodily fluid. In certain aspects, the sample may include or may be derived from a tissue sample, biopsy, resection or isolated cells from said individual.

In certain aspects, the sample is a tissue sample. The sample may be a sample of tumor tissue, such as cancerous tumor tissue. The sample may have been obtained by a tumor biopsy. In some aspects, the sample is a lymphoid tissue sample, such as a lymphoid lesion sample or lymph node biopsy. In some cases, the sample is a skin biopsy.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of a individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of CD19 protein or nucleic acid in the blood, in white blood cells, peripheral blood mononuclear cells, granulocytes and/or red blood cells.

The sample may be fresh or archival. For example, archival tissue may be from the first diagnosis of an individual, or a biopsy at a relapse. In certain aspects, the sample is a fresh biopsy.

### Individual Status

The individual may be an animal, mammal, a placental mammal, a marsupial (e.g., kangaroo, wombat), a monotreme (e.g., duckbilled platypus), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the individual may be any of its forms of development, for example, a foetus. In one preferred embodiment, the individual is a human. The terms “subject”, “patient” and “individual” are used interchangeably herein.

In some aspects disclosed herein, an individual has, or is suspected as having, or has been identified as being at risk of, cancer. In some aspects disclosed herein, the individual has already received a diagnosis of cancer. The individual may have received a diagnosis of non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenstrom Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

In some cases, the individual has received a diagnosis of non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL) [Fielding A., Haematologica. 2010 January; 95 (1): 8-12].

In some cases, the individual has received a diagnosis of a solid cancer containing CD19+ expressing infiltrating cells.

The Individual may be undergoing, or have undergone, a therapeutic treatment for that cancer. The subject may, or may not, have previously received ADCX19. In some cases the cancer is lymphoma, including non-Hodgkins lymphoma.

The Individual may be undergoing, or have undergone, treatment with an anti-CD79b agent. In some cases the individual may be refractory to treatment (or further treatment) with the anti-CD79b agent. In some cases the anti-CD79b agent may be Polatuzumab vedotin. In embodiments where the individual is undergoing, or has undergone, treatment with an anti-CD79b agent, the anti-CD19 ADC may be administered in combination with an anti-CD79b agent, or without continued administration of the anti-CD79b agent.

### Controls

In some aspects, target expression in the individual is compared to target expression in a control. Controls are useful to support the validity of staining, and to identify experimental artefacts.

In some cases, the control may be a reference sample or reference dataset. The reference may be a sample that has been previously obtained from a individual with a known degree of suitability. The reference may be a dataset obtained from analyzing a reference sample.

Controls may be positive controls in which the target molecule is known to be present, or expressed at high level, or negative controls in which the target molecule is known to be absent or expressed at low level.

Controls may be samples of tissue that are from individuals who are known to benefit from the treatment. The tissue may be of the same type as the sample being tested. For example, a sample of tumor tissue from a individual may be compared to a control sample of tumor tissue from a individual who is known to be suitable for the treatment, such as a individual who has previously responded to the treatment.

In some cases the control may be a sample obtained from the same individual as the test sample, but from a tissue known to be healthy. Thus, a sample of cancerous tissue from a individual may be compared to a non-cancerous tissue sample.

In some cases, the control is a cell culture sample.

In some cases, a test sample is analyzed prior to incubation with an antibody to determine the level of background staining inherent to that sample.

In some cases an isotype control is used. Isotype controls use an antibody of the same class as the target specific antibody, but are not immunoreactive with the sample. Such controls are useful for distinguishing non-specific interactions of the target specific antibody.

The methods may include hematopathologist interpretation of morphology and immunohistochemistry, to ensure accurate interpretation of test results. The method may involve confirmation that the pattern of expression correlates with the expected pattern. For example, where the amount of CD19 and/or CD79b expression is analyzed, the method may involve confirmation that in the test sample the expression is observed as membrane staining, with a cytoplasmic component. The method may involve confirmation that the ratio of target signal to noise is above a threshold level, thereby allowing clear discrimination between specific and non-specific background signals.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

Disclosed herein are methods of therapy. Also provided is a method of treatment, comprising administering to a subject in need of treatment a therapeutically-effective amount of an anti-CD19 ADC and an anti-CD79b agent. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The anti-CD19 ADC comprises an anti-CD19 antibody. The anti-CD19 antibody may be RB4v1.2 antibody. The ADC may comprise a drug which is a PBD dimer. The ADC may be ADCx19. The ADC may be an ADC disclosed in WO2014/057117.

The anti-CD25 ADC comprises an anti-CD25 antibody. The anti-CD25 antibody may be HuMax-TAC™. The ADC may comprise a drug which is a PBD dimer. The ADC may be an anti-CD25-ADC, and in particular, ADCX25 or camidanlumab tesirine. The ADC may be an ADC disclosed in WO2014/057119.

The anti-CD22 ADC comprises an anti-CD22 antibody. The anti-CD22 antibody may be EMabC220. The ADC may comprise a drug which is a PBD dimer. The ADC may be an anti-CD22-ADC such as ADCT-602 or ADCx22. The ADC may be an ADC disclosed in WO2014/057122 or WO2016/166307.

Typically the an individual to which the treatment disclosed herein is administered is in need of said treatment, or has been identified or diagnosed as in need of the treatment.

The anti-CD79b agent may be Polatuzumab vedotin.

The treatment may involve administration of the anti-CD19 ADC/anti-CD79b agent combination alone or in further combination with other treatments, either simultaneously or sequentially dependent upon the condition to be treated.

An example method of treatment involves:
(1) identifying an individual has been treated with, or is being treated with an anti-CD79b agent, such as Polatuzumab vedotin;
(2) administering to the individual an anti-CD19 ADC, such as ADCx19; and, optionally
(3) administering to the individual an anti-CD79b agent, such as Polatuzumab vedotin in combination with the anti-CD19 ADC (for example, at the same time as the ADC, or after the ADC).

Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, antimetabolites, spindle poison plant alkaloids, cytotoxic/antitumor antibiotics, topoisomerase inhibitors, antibodies, photosensitizers, and kinase inhibitors. Chemotherapeutic agents include compounds used in “targeted therapy” and conventional chemotherapy.

Examples of chemotherapeutic agents include: Lenalidomide (REVLIMID®, Celgene), Vorinostat (ZOLINZAR, Merck), Panobinostat (FARYDAK®, Novartis), Mocetinostat (MGCD0103), Everolimus (ZORTRESS®, CERTICAN®, Novartis), Bendamustine (TREAKISYM®, RIBOMUSTIN®, LEVACT®, TREANDAR, Mundipharma International), erlotinib (TARCEVA®, Genentech/OSI Pharm.), docetaxel (TAXOTERER, Sanofi-Aventis), 5-FU (fluorouracil, 5-fluorouracil, CAS No. 51-21-8), gemcitabine (GEMZAR®, Lilly), PD-0325901 (CAS No. 391210-10-9, Pfizer), cisplatin (cis-diamine, dichloroplatinum (II), CAS No. 15663-27-1), carboplatin (CAS No. 41575-94-4), paclitaxel (TAXOL®, Bristol-Myers Squibb Oncology, Princeton, N.J.), trastuzumab b (HERCEPTIN®, Genentech), temozolomide (4-methyl-5-oxo-2,3,4,6,8-pentazabicyclo[4.3.0]nona-2,7,9-triene-9-carboxamide, CAS No. 85622-93-1, TEMODAR®, TEMODAL®, Schering Plough), tamoxifen ((Z)-2-[4-(1,2-diphenylbut-1-enyl) phenoxy]-N,N-dimethylethanamine, NOLVADEX®, ISTUBAL®, VALODEX®), and doxorubicin (ADRIAMYCIN®), Akti-1/2, HPPD, and rapamycin.

More examples of chemotherapeutic agents include: oxaliplatin (ELOXATIN®, Sanofi), bortezomib (VELCADE®, Millennium Pharm.), sutent (SUNITINIB®, SU11248, Pfizer), letrozole (FEMARA®, Novartis), imatinib mesylate (GLEEVEC®, Novartis), XL-518 (Mek inhibitor, Exelixis, WO 2007/044515), ARRY-886 (Mek inhibitor, AZD6244, Array BioPharma, Astra Zeneca), SF-1126 (anti-CD79b agent, Semafore Pharmaceuticals), BEZ-235 (anti-CD79b agent, Novartis), XL-147 (anti-CD79b agent, Exelixis), PTK787/ZK 222584 (Novartis), fulvestrant (FASLODEX®, AstraZeneca), leucovorin (folinic acid), rapamycin (sirolimus, RAPAMUNE®, Wyeth), lapatinib (TYKERBR, GSK572016, Glaxo Smith Kline), lonafarnib (SARASAR™, SCH 66336, Schering Plough), sorafenib (NEXAVAR®, BAY43-9006, Bayer Labs), gefitinib (IRESSA®, AstraZeneca), irinotecan (CAMPTOSAR®, CPT-11, Pfizer), tipifarnib (ZARNESTRA™, Johnson & Johnson), ABRAXANE™ (Cremophor-free), albumin-engineered nanoparticle formulations of paclitaxel (American Pharmaceutical Partners, Schaumberg, II), vandetanib (rINN, ZD6474, ZACTIMA®, AstraZeneca), chloranmbucil, AG1478, AG1571 (SU 5271; Sugen), temsirolimus (TORISEL®, Wyeth), pazopanib (GlaxoSmithKline), canfosfamide (TELCYTA®, Telik), thiotepa and cyclosphosphamide (CYTOXAN®, NEOSAR®); alkyl sulfonates such as busulfan, improsulfan and piposulfan; aziridines such as benzodopa, carboquone, meturedopa, and uredopa; ethylenimines and methylamelamines including altretamine, triethylenemelamine, triethylenephosphoramide, triethylenethio phosphoramide and trimethylomelamine; acetogenins (especially bullatacin and bullatacinone); a camptothecin (including the synthetic analog topotecan); bryostatin; callystatin; CC-1065 (including its adozelesin, carzelesin and bizelesin synthetic analogs); cryptophycins (particularly cryptophycin 1 and cryptophycin 8); dolastatin; duocarmycin (including the synthetic analogs, KW-2189 and CB1-TM1); eleutherobin; pancratistatin; a sarcodictyin; spongistatin; nitrogen mustards such as chlorambucil, chlornaphazine, chlorophosphamide, estramustine, ifosfamide, mechlorethamine, mechlorethamine oxide hydrochloride, melphalan, novembichin, phenesterine, prednimustine, trofosfamide, uracil mustard; nitrosoureas such as carmustine, chlorozotocin, fotemustine, lomustine, nimustine, and ranimnustine; antibiotics such as the enediyne antibiotics (e.g. calicheamicin, calicheamicin gamma11, calicheamicin omega11 (Angew Chem. Intl. Ed. Engl. (1994) 33:183-186); dynemicin, dynemicin A; bisphosphonates, such as clodronate; an esperamicin; as well as neocarzinostatin chromophore and related chromoprotein enediyne antibiotic chromophores), aclacinomysins, actinomycin, authramycin, azaserine, bleomycins, cactinomycin, carabicin, carminomycin, carzinophilin, chromomycinis, dactinomycin, daunorubicin, detorubicin, 6-diazo-5-oxo-L-norleucine, morpholino-doxorubicin, cyanomorpholino-doxorubicin, 2-pyrrolino-doxorubicin and deoxydoxorubicin), epirubicin, esorubicin, idarubicin, nemorubicin, marcellomycin, mitomycins such as mitomycin C, mycophenolic acid, nogalamycin, olivomycins, peplomycin, porfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogs such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine; androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; eniluracil; amsacrine; bestrabucil; bisantrene; edatraxate; defofamine; demecolcine; diaziquone; elfornithine; elliptinium acetate; an epothilone; etoglucid; gallium nitrate; hydroxyurea; lentinan; lonidainine; maytansinoids such as maytansine and ansamitocins; mitoguazone; mitoxantrone; mopidanmol; nitraerine; pentostatin; phenamet; pirarubicin; losoxantrone; podophyllinic acid; 2-ethylhydrazide; procarbazine; PSK® polysaccharide complex (JHS Natural Products, Eugene, OR); razoxane; rhizoxin; sizofiran; spirogermanium; tenuazonic acid; triaziquone; 2,2′,2″-trichlorotriethylamine; trichothecenes (especially T-2 toxin, verracurin A, roridin A and anguidine); urethan; vindesine; dacarbazine; mannomustine; mitobronitol; mitolactol; pipobroman; gacytosine; arabinoside (“Ara-C”); cyclophosphamide; thiotepa; 6-thioguanine; mercaptopurine; methotrexate; platinum analogs such as cisplatin and carboplatin; vinblastine; etoposide (VP-16); ifosfamide; mitoxantrone; vincristine; vinorelbine (NAVELBINE®); novantrone; teniposide; edatrexate; daunomycin; aminopterin; capecitabine (XELODAR, Roche); ibandronate; CPT-11; topoisomerase inhibitor RFS 2000; difluoromethylornithine (DMFO); retinoids such as retinoic acid; and pharmaceutically acceptable salts, acids and derivatives of any of the above. Combinations of agents may be used, such as CHP (doxorubicin, prednisone, cyclophosphamide), or CHOP (doxorubicin, prednisone, cyclophopsphamide, vincristine).

Also included in the definition of “chemotherapeutic agent” are: (i) anti-hormonal agents that act to regulate or inhibit hormone action on tumors such as anti-estrogens and selective estrogen receptor modulators (SERMs), including, for example, tamoxifen (including NOLVADEX®; tamoxifen citrate), raloxifene, droloxifene, 4-hydroxytamoxifen, trioxifene, keoxifene, LY117018, onapristone, and FARESTON® (toremifine citrate); (ii) aromatase inhibitors that inhibit the enzyme aromatase, which regulates estrogen production in the adrenal glands, such as, for example, 4 (5)-imidazoles, aminoglutethimide, MEGASER (megestrol acetate), AROMASIN® (exemestane; Pfizer), formestanie, fadrozole, RIVISOR® (vorozole), FEMARA® (letrozole; Novartis), and ARIMIDEX® (anastrozole; AstraZeneca); (iii) anti-androgens such as flutamide, nilutamide, bicalutamide, leuprolide, and goserelin; as well as troxacitabine (a 1,3-dioxolane nucleoside cytosine analog); (iv) protein kinase inhibitors such as MEK inhibitors (WO 2007/044515); (v) lipid kinase inhibitors; (vi) antisense oligonucleotides, particularly those which inhibit expression of genes in signaling pathways implicated in aberrant cell proliferation, for example, PKC-alpha, Raf and H-Ras, such as oblimersen (GENASENSE®, Genta Inc.); (vii) ribozymes such as VEGF expression inhibitors (e.g., ANGIOZYMER) and HER2 expression inhibitors; (viii) vaccines such as gene therapy vaccines, for example, ALLOVECTIN®, LEUVECTIN®, and VAXID®; PROLEUKIN® rIL-2; topoisomerase 1 inhibitors such as LURTOTECAN®; ABARELIX® rmRH; (ix) anti-angiogenic agents such as bevacizumab (AVASTIN®, Genentech); and pharmaceutically acceptable salts, acids and derivatives of any of the above.

Also included in the definition of “chemotherapeutic agent” are therapeutic antibodies such as alemtuzumab (Campath), bevacizumab (AVASTIN®, Genentech); cetuximab (ERBITUX®, Imclone); panitumumab (VECTIBIX®, Amgen), pertuzumab (PERJETA™, OMNITARG™, 2C₄, Genentech), trastuzumab (HERCEPTIN®, Genentech), MDX-060 (Medarex) and the antibody drug conjugate, gemtuzumab ozogamicin (MYLOTARG®, Wyeth).

Humanized monoclonal antibodies with therapeutic potential as chemotherapeutic agents in combination with the conjugates of the disclosure include: alemtuzumab, apolizumab, aselizumab, atlizumab, bapineuzumab, bevacizumab, bivatuzumab mertansine, cantuzumab mertansine, cedelizumab, certolizumab pegol, cidfusituzumab, cidtuzumab, daclizumab, eculizumab, efalizumab, epratuzumab, erlizumab, felvizumab, fontolizumab, gemtuzumab ozogamicin, inotuzumab ozogamicin, ipilimumab, labetuzumab, lintuzumab, matuzumab, mepolizumab, motavizumab, motovizumab, natalizumab, nimotuzumab, nolovizumab, numavizumab, omalizumab, palivizumab, pascolizumab, pecfusituzumab, pectuzumab, pertuzumab, pexelizumab, ralivizumab, ranibizumab, reslivizumab, reslizumab, resyvizumab, rovelizumab, ruplizumab, sibrotuzumab, siplizumab, sontuzumab, tacatuzumab tetraxetan, tadocizumab, talizumab, tefibazumab, tocilizumab, toralizumab, trastuzumab, tucotuzumab celmoleukin, tucusituzumab, umavizumab, urtoxazumab, and visilizumab.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which may be oral, or by injection, e.g. cutaneous, subcutaneous, or intravenous.

Pharmaceutical compositions for oral administration may be in tablet, capsule, powder or liquid form. A tablet may comprise a solid carrier or an adjuvant. Liquid pharmaceutical compositions generally comprise a liquid carrier such as water, petroleum, animal or vegetable oils, mineral oil or synthetic oil. Physiological saline solution, dextrose or other saccharide solution or glycols such as ethylene glycol, propylene glycol or polyethylene glycol may be included. A capsule may comprise a solid carrier such a gelatin.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Dosage

It will be appreciated by one of skill in the art that appropriate dosages of the anti-CD19 ADC and/or the anti-CD79b agent, and compositions comprising these active elements, can vary from subject to subject. Determining the optimal dosage will generally involve the balancing of the level of therapeutic benefit against any risk or deleterious side effects. The selected dosage level will depend on a variety of factors including, but not limited to, the activity of the particular compound, the route of administration, the time of administration, the rate of excretion of the compound, the duration of the treatment, other drugs, compounds, and/or materials used in combination, the severity of the condition, and the species, sex, age, weight, condition, general health, and prior medical history of the subject. The amount of compound and route of administration will ultimately be at the discretion of the physician, veterinarian, or clinician, although generally the dosage will be selected to achieve local concentrations at the site of action which achieve the desired effect without causing substantial harmful or deleterious side-effects.

In certain aspects, the dosage of anti-CD19 ADC is determined by the expression of CD19 observed in a sample obtained from the subject. Thus, the level or localisation of expression of CD19 in the sample may be indicative that a higher or lower dose of anti-CD19 ADC is required. For example, a high expression level of CD19 may indicate that a higher dose of anti-CD19 ADC would be suitable. In some cases, a high expression level of CD19 may indicate the need for administration of another agent in addition to the anti-CD19 ADC. For example, administration of the anti-CD19 ADC in conjunction with a chemotherapeutic agent. A high expression level of CD19 may indicate a more aggressive therapy.

In certain aspects, the dosage of the anti-CD79b agent is determined by the expression of observed in a sample obtained from the subject. Thus, the level or localisation of expression of in the sample may be indicative that a higher or lower dose of anti-CD79b agent is required. For example, a high expression level of CD79b may indicate that a higher dose of anti-CD79b agent would be suitable. In some cases, a high expression level of CD79b may indicate the need for administration of another agent in addition to the anti-CD79b agent. For example, administration of the anti-CD79b agent in conjunction with a chemotherapeutic agent. A high expression level of CD79b may indicate a more aggressive therapy.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of each active compound is in the range of about 100 ng to about 25 mg (more typically about 1 μg to about 10 mg) per kilogram body weight of the subject per day. Where the active compound is a salt, an ester, an amide, a prodrug, or the like, the amount administered is calculated on the basis of the parent compound and so the actual weight to be used is increased proportionately.

In one embodiment, each active compound is administered to a human subject according to the following dosage regime: about 100 mg, 3 times daily.

In one embodiment, each active compound is administered to a human subject according to the following dosage regime: about 150 mg, 2 times daily.

In one embodiment, each active compound is administered to a human subject according to the following dosage regime: about 200 mg, 2 times daily.

However in one embodiment, each conjugate compound is administered to a human subject according to the following dosage regime: about 50 or about 75 mg, 3 or 4 times daily.

In one embodiment, each conjugate compound is administered to a human subject according to the following dosage regime: about 100 or about 125 mg, 2 times daily.

For the anti-CD19 ADC, where it is a PBD bearing ADC, the dosage amounts described above may apply to the conjugate (including the PBD moiety and the linker to the antibody) or to the effective amount of PBD compound provided, for example the amount of compound that is releasable after cleavage of the linker.

The anti-CD19 ADC comprises an anti-CD19 antibody. The anti-CD19 antibody may be RB4v1.2 antibody. The ADC may comprise a drug which is a PBD dimer. The anti-CD19-ADC may be ADCx19. The anti-CD19 ADC may be Loncastuximab tesirine. The ADC may be an ADC disclosed in WO2014/057117.

The anti-CD79b agent may be Polatuzumab vedotin.

### Antibodies

The term “antibody” herein is used in the broadest sense and specifically covers monoclonal antibodies, polyclonal antibodies, dimers, multimers, multispecific antibodies (e.g., bispecific antibodies), intact antibodies (also described as “full-length” antibodies) and antibody fragments, so long as they exhibit the desired biological activity, for example, the ability to bind CD19 (Miller et al (2003) Jour. of Immunology 170:4854-4861). Antibodies may be murine, human, humanized, chimeric, or derived from other species such as rabbit, goat, sheep, horse or camel.

### BRIEF DESCRIPTION OF THE FIGURES

Embodiments and experiments illustrating the principles of the disclosure will now be discussed with reference to the accompanying FIGURES in which:

FIG. 1. Plot of median tumour volume showing in vivo efficacy of a ADCx19 and Pola-V combination

The disclosure includes the combination of the aspects and preferred features described except where such a combination is clearly impermissible or expressly avoided.

The section headings used herein are for organizational purposes only and are not to be construed as limiting the subject matter described.

Aspects and embodiments of the present disclosure will now be illustrated, by way of example, with reference to the accompanying FIGURES. Further aspects and embodiments will be apparent to those skilled in the art. All documents mentioned in this text are incorporated herein by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

As used in the specification and the appended claims, the singular forms “a,” “an,” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

### STATEMENTS OF INVENTION

1. A method for treating a disorder in an individual, the method comprising administering to the individual an effective amount of an anti-CD19 ADC (or anti-CD22 ADC or anti-CD25 ADC) and an anti-CD79b agent.
2. The method according to statement 1, wherein the individual is selected for treatment.
3. The method according to statement 2, wherein the individual is selected for treatment with an anti-CD19 ADC (or anti-CD22 ADC or anti-CD25 ADC) if the individual has been treated with an anti-CD79b agent.
4. The method according to statement 2, where the individual is selected for treatment with an anti-CD19 ADC (or anti-CD22 ADC or anti-CD25 ADC) if the individual is being treated with an anti-CD79b agent.
5. The method according to any one of the preceding statements, wherein the individual is selected for treatment if the individual is refractory to treatment, or further treatment, with the anti-CD79b agent.
6. A method for treating a disorder in an individual, the method comprising:
   (i) selecting an individual as suitable for treatment by a method according to any one of statements 3 to 5; and
   (ii) administering to the individual an effective amount of an anti-CD19 ADC (or anti-CD22 ADC or anti-CD25 ADC).
7. The method according to statement 6, further comprising administering an anti-CD79b agent in combination with the anti-CD19 ADC (or anti-CD22 ADC or anti-CD25 ADC).
8. The method according to any one of statements 1 to 5 or 7, wherein the treatment comprises administering the anti-CD19 ADC (or anti-CD22 ADC or anti-CD25 ADC) before the anti-CD79b agent, simultaneous with the anti-CD79b agent, or after the anti-CD79b agent.
9. The method according to any preceding statement, wherein the treatment further comprises administering a chemotherapeutic agent.
10. The method according to any preceding statement, wherein the individual is human.
11. The method according to any preceding statement, wherein the individual has a disorder or has been determined to have a disorder.
12. The method according to statement 11, wherein the individual has, or has been determined to have, a cancer which expresses CD19 (or CD22 or CD25) or CD19+ve (or CD22+ve or CD25+ve) tumour-associated non-tumour cells, such as CD19+ve (or CD22+ve or CD25+ve) infiltrating cells.
13. The method according to any preceding statement, wherein the individual is undergoing treatment with an anti-CD79b agent.
14. The method according to any preceding statement, wherein the individual has undergone treatment with an anti-CD79b agent.
15. The method according to any preceding statement, wherein the individual is refractory to treatment, or further treatment, with the anti-CD79b agent.
16. The method according to any one of the preceding statements, wherein the treatment has increased efficacy as compared to monotherapy with either an anti-CD19 ADC (or anti-CD22 ADC or anti-CD25 ADC) or an anti-CD79b agent alone.
17. The method according to any preceding statement, wherein the disorder is a proliferative disease.
18. The method of statement 17, wherein the disorder is characterised by the presence of a neoplasm comprising CD19+ve cells (or CD22+ve cells or CD25+ve cells).
19. The method of statement 17, wherein the individual has, or has been has been determined to have, a disorder characterised by the presence of a neoplasm comprising both CD25+ve and CD25−ve cells.
20 The method of statement 17, wherein the individual has, or has been has been determined to have, a disorder characterised by the presence of a neoplasm comprising, or composed of, CD25−ve neoplastic cells.
21. The method according to any of statements 18 to 20, wherein the neoplasm is all or part of a solid tumour.
22. The method of statement 21, wherein the solid tumour is associated with CD25+ve infiltrating cells;
   optionally wherein the solid tumour is associated with high levels of CD25+ve infiltrating cells.
23. The method of statement 21 or 122, wherein the solid tumour is selected from the group consisting of pancreatic cancer, breast cancer (including triple negative breast cancer), colorectal cancer, gastric and oesophageal cancer, melanoma, non-small cell lung cancer, ovarian cancer, hepatocellular carcinoma, renal cell carcinoma, bladder, and head and neck cancer.
24. The method of any preceding statement, wherein the disorder is cancer.
25. The method of any preceding statement, wherein the disorder is selected from the group comprising: non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).
26. The method of any preceding statement, wherein the anti-CD79b agent is an anti-CD79b ADC.
27. The method of statement 26, wherein the CD79b ADC is a conjugate of formula (I):

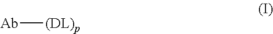

wherein:
Ab is an antibody that binds to CD79b;
DL is

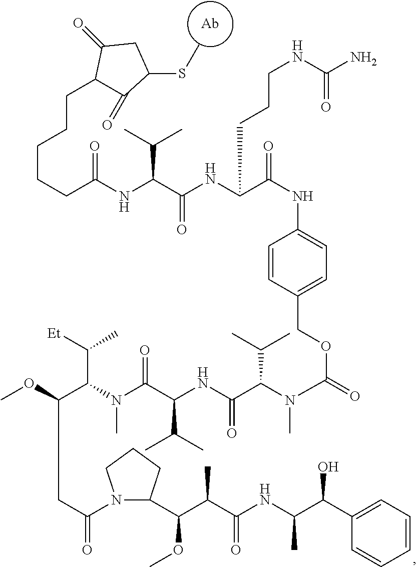 [SMILES: CC[C@H](C)[C@@H]([C@@H](CC(=O)N1CCCC1[C@H](OC)[C@@H](C)C(=O)N[C@H](C)[C@@H](O)c1ccccc1)OC)N(C)C(=O)[C@@H](NC(=O)[C@H](C(C)C)N(C)C(=O)OCc1ccc(NC(=O)[C@H](CCCNC(N)=O)NC(=O)[C@@H](NC(=O)CCCCCC2C(=O)CC(SC)C2=O)C(C)C)cc1)C(C)C]

and p is between 1 and 8, such as between 3 and 4, for example about 3.5.
28. The method of any preceding statement, wherein the anti-CD79b agent or ADC comprises an antibody having a VH domain comprising a VH CDR1, a VH CDR2, and a VH CDR3, wherein the antibody comprises the CDR sequences of the VH domain having the sequence according to SEQ ID NO: 17.
29. The method of statement 28, wherein the antibody further comprises a VL domain comprising a VL CDR1, a VL CDR2, and a VL CDR3, wherein the antibody comprises the CDR sequences of the VL domain having the sequence according to SEQ ID NO: 18.
30. The method of any one of statements 1 to 27, wherein the anti-CD79b agent or ADC comprises an antibody having a VH domain and a VL domain,
   the VH domain having a VH CDR1 with the amino acid sequence of SEQ ID NO. 19, a VH CDR2 with the amino acid sequence of SEQ ID NO. 20, and a VH CDR3 with the amino acid sequence of SEQ ID NO. 21; and
   the VL domain having a VL CDR1 with the amino acid sequence of SEQ ID NO. 22, a VL CDR2 with the amino acid sequence of SEQ ID NO. 23, and a VL CDR3 with the amino acid sequence of SEQ ID NO. 24.
31. The method of any one of statements 1 to 27, wherein the anti-CD79b agent or ADC comprises an antibody having a VH domain having the sequence according to SEQ ID NO: 17, and a VL domain having the sequence according to SEQ ID NO: 18.
32. The method of any one of statements 1 to 27, wherein the anti-CD79b agent is polatuzumab vedotin.
33. The method according to any preceding statement, wherein the anti-CD19 ADC (or anti-CD22 ADC or anti-CD25 ADC) comprises a conjugate of formula L-(DL) p, where DL is of formula I or II:

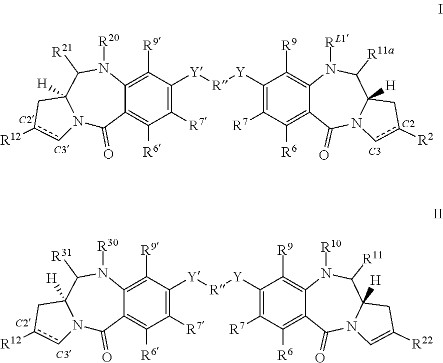

wherein:
L is an antibody (Ab) which is an antibody that binds to CD19 (or CD22 or CD25);
   when there is a double bond present between C2′ and C3′, R¹² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

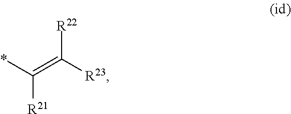

wherein each of R²¹, R²² and R²³ are independently selected from H, C₁₋₃ saturated alkyl, C₂₋₃ alkenyl, C₂₋₃ alkynyl and cyclopropyl, where the total number of carbon atoms in the R¹² group is no more than 5;

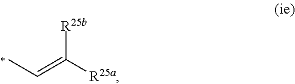

wherein one of R^{25a} and R^{25b} is H and the other is selected from: phenyl, which phenyl is optionally substituted by a group selected from halo, methyl, methoxy; pyridyl; and thiophenyl; and

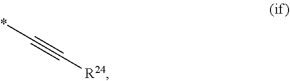

where R²⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2′ and C3′,
R¹² is

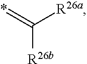

where R^{26a} and R^{26b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{26a} and R^{26b} is H, the other is selected from nitrile and a C₁₋₄ alkyl ester;
R⁶ and R⁹ are independently selected from H, R, OH, OR, SH, SR, NH₂, NHR, NRR′, nitro, Me₃Sn and halo;
where R and R′ are independently selected from optionally substituted C₁₋₁₂ alkyl, C₃₋₂₀ heterocyclyl and C₅₋₂₀ aryl groups;
R⁷ is selected from H, R, OH, OR, SH, SR, NH₂, NHR, NHRR′, nitro, Me₃Sn and halo;
R″ is a C₃₋₁₂ alkylene group, which chain may be interrupted by one or more heteroatoms, e.g. O, S, NR^{N2} (where R^{N2} is H or C₁₋₄ alkyl), and/or aromatic rings, e.g. benzene or pyridine;
Y and Y′ are selected from O, S, or NH;
R^{6′}, R^{7′}, R^{9′} are selected from the same groups as R⁶, R⁷ and R⁹ respectively;

### [Formula I]

R^{L1′} is a linker for connection to the antibody (Ab);
R^{11a} is selected from OH, ORA, where RA is C₁₋₄ alkyl, and SOM, where z is 2 or 3 and M is a monovalent pharmaceutically acceptable cation;
R²⁰ and R²¹ either together form a double bond between the nitrogen and carbon atoms to which they are bound or;
R²⁰ is selected from H and RC, where RC is a capping group;
R²¹ is selected from OH, ORA and SO₂M;
when there is a double bond present between C2 and C3, R² is selected from the group consisting of:
(ia) C₅₋₁₀ aryl group, optionally substituted by one or more substituents selected from the group comprising: halo, nitro, cyano, ether, carboxy, ester, C₁₋₇ alkyl, C₃₋₇ heterocyclyl and bis-oxy-C₁₋₃ alkylene;
(ib) C₁₋₅ saturated aliphatic alkyl;
(ic) C₃₋₆ saturated cycloalkyl;

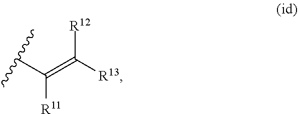

wherein each of R¹¹, R¹² and R¹³ are independently selected from H, C₁₋₃ saturated alkyl, C₂₋₃ alkenyl, C₂₋₃ alkynyl and cyclopropyl, where the total number of carbon atoms in the R² group is no more than 5;

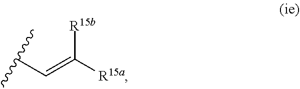

wherein one of R^{15a} and R^{15b} is H and the other is selected from: phenyl, which phenyl is optionally substituted by a group selected from halo, methyl, methoxy; pyridyl; and thiophenyl; and

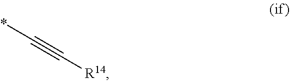

where R¹⁴ is selected from: H; C₁₋₃ saturated alkyl; C₂₋₃ alkenyl; C₂₋₃ alkynyl; cyclopropyl; phenyl, which phenyl is optionally substituted by a group selected from halo, methyl, methoxy; pyridyl; and thiophenyl;
when there is a single bond present between C2 and C3,
R² is

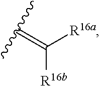

where R^{16a} and R^{16b} are independently selected from H, F, C₁₋₄ saturated alkyl, C₂₋₃ alkenyl, which alkyl and alkenyl groups are optionally substituted by a group selected from C₁₋₄ alkyl amido and C₁₋₄ alkyl ester; or, when one of R^{16a} and R^{16b} is H, the other is selected from nitrile and a C₁₋₄ alkyl ester;

### [Formula II]

R²² is of formula IIIa, formula IIIb or formula IIIc:

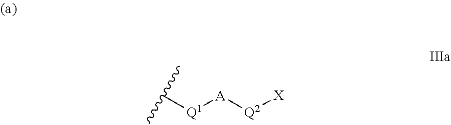

where A is a C₅₋₇ aryl group, and either
(i) Q¹ is a single bond, and Q² is selected from a single bond and —Z—(CH₂)ₙ—, where Z is selected from a single bond, O, S and NH and n is from 1 to 3; or
(ii) Q¹ is —CH═CH—, and Q² is a single bond;

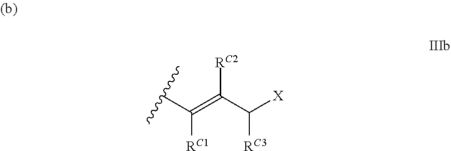

where;
R^{C1}, R^{C2} and R^{C3} are independently selected from H and unsubstituted C₁₋₂ alkyl;

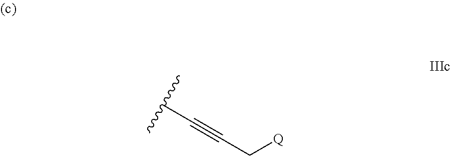

where Q is selected from O—R^{L2′}, S—R^{L2′} and NR^{N}—R^{L2′}, and R^{N} is selected from H, methyl and ethyl
X is selected from the group comprising: O—R^{L2′}, S—R^{L2′}, CO₂—R^{L2′}, CO—R^{L2′}, NH—C(═O)—R^{L2}, NHNH—R^{L2′}, CONHNH—R^{L2′},

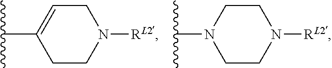

NR^{N}R^{L2′}, wherein R^{N} is selected from the group comprising H and C₁₋₄ alkyl;
R^{L2′} is a linker for connection to the antibody (Ab);
R¹⁰ and R¹¹ either together form a double bond between the nitrogen and carbon atoms to which they are bound or;
R¹⁰ is H and R¹¹ is selected from OH, ORA and SO₂M;
R³⁰ and R³¹ either together form a double bond between the nitrogen and carbon atoms to which they are bound or;
R³⁰ is H and R³¹ is selected from OH, ORA and SO₂M.
34. The method according to statement 33, wherein the anti-CD19 ADC (or anti-CD22 ADC or anti-CD25 ADC) has the chemical structure:

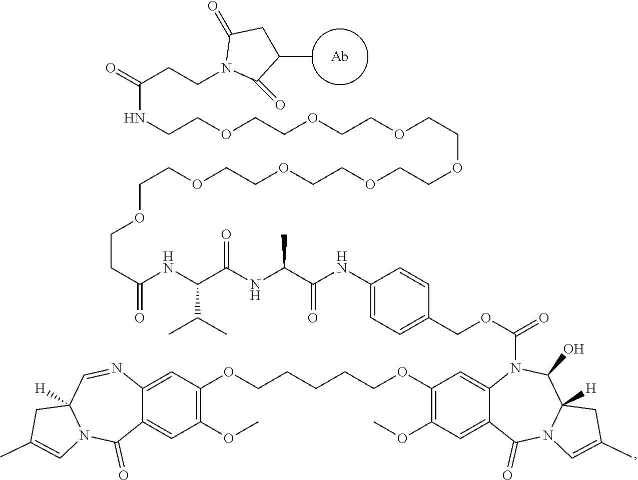

where Ab is the antibody that binds to CD19 (or CD22 or CD25), and p is between 1 and 8.
35. The method of any preceding statement, wherein the anti-CD19 ADC comprises an antibody having a VH domain comprising a VH CDR1, a VH CDR2, and a VH CDR3, wherein the antibody comprises the CDR sequences of the VH domain having the sequence according to SEQ ID NO: 2.
36. The method of statement 35, wherein the antibody further comprises a VL domain comprising a VL CDR1, a VL CDR2, and a VL CDR3, wherein the antibody comprises the CDR sequences of the VL domain having the sequence according to SEQ ID NO: 8.
37. The method of any preceding statement, wherein the anti-CD19 ADC comprises an antibody having a VH domain having the sequence according to SEQ ID NO: 2.
38. The method of statement 37, wherein the anti-CD19 ADC further comprises an antibody having a VH domain having the sequence according to SEQ ID NO: 8.
39. The method according to any previous statement, wherein the anti-CD19 ADC comprises a heavy chain having the amino acid sequence of SEQ ID NO. 13 and/or a light chain having the amino acid sequence of SEQ ID NO. 14.
40. The method according to any preceding statement, wherein the anti-CD19 ADC is ADCx19.
41. The method according to any one of statements 1 to 39, wherein the anti-CD19 ADC is Loncastuximab tesirine
42. An anti-CD19 ADC (or anti-CD22 ADC or anti-CD25 ADC) for use in a method of treatment according to any preceding statement.
43. A composition comprising an anti-CD19 ADC (or anti-CD22 ADC or anti-CD25 ADC), for use in a method of treatment according to any preceding statement.
44. A composition comprising an anti-CD19 ADC (or anti-CD22 ADC or anti-CD25 ADC) and an anti-CD79b agent.
45. The anti-CD19 ADC (or anti-CD22 ADC or anti-CD25 ADC) for use according to statement 42, the composition for use according to statement 43, or the composition according to statement 44, wherein the anti-CD19 ADC is as defined in any one of statements 33 to 39 and/or the anti-CD79b agent is as defined in any one of statements 27 to 32.
46. An anti-CD79b agent for use in a method of treatment according to any preceding statement.
47. A composition comprising an anti-CD79b agent, for use in a method of treatment according to any preceding statement.
48. The anti-CD79b agent for use according to statement 46, or the composition for use according to statement 42, wherein the anti-CD79b agent is polatuzumab vedotin.
49. Use of an anti-CD19 ADC (or anti-CD22 ADC or anti-CD25 ADC) in the manufacture of a medicament for treating a disorder in an individual, wherein the treatment comprises the method of any preceding statement.
50. The use of an anti-CD19 ADC according to statement 49, wherein the anti-CD19 ADC is as defined in any one of statements 33 to 39.
51. Use of an anti-CD79b agent in the manufacture of a medicament for treating a disorder in an individual, wherein the treatment comprises the method of any preceding statement.
52. The use of the anti-CD79b agent according to statement 51, wherein the anti-CD79b agent is polatuzumab vedotin.
53. A kit comprising:
   a first medicament comprising an anti-CD19 ADC (or anti-CD22 ADC or anti-CD25 ADC);
   a package insert comprising instructions for administration of the first medicament according to the method of any one of statements 1 to 41.
54. The kit according to statement 53, wherein the anti-CD19 ADC is as defined in any one of statements 28 to 36.
55. The kit according to statements 53 or 54, further comprising:
   a second medicament comprising an anti-CD79b agent.
56. The kit according to statement 55, wherein the anti-CD79b agent is polatuzumab vedotin.

### EXAMPLES

### Example 1: In Vitro Efficacy of a ADCx19 and Pola-V Combination

### Methods

MTT proliferation assay on cell lines exposed (120 h) to increasing ADCx19 and Pola-V concentrations. Synergy at 120 h was assessed by Chou-Talalay combination index (CI) (synergism CI<0.9, additive CI=0.9-1.1, antagonism/no benefit CI>1.1) on an activated B cell like (ABC) DLBCL cell line (TMD8), a germinal center (GCB) DLBCL (WSU-DLCL2) cell line, and a Burkitt lymphoma (Ramos) cell line.

### Results

ADCx19 was combined with the anti-CD79b agent, Polatuzumab vedotin (Pola-V) in GCB- and ABC-DLBCL cell lines, as well as a Burkitt lymphoma cell line. Synergism was achieved in Ramos and TMD8 cell lines combining ADCx19 with Pola-V (median CI 0.74 and 0.764, respectively), with WSU-DLCL2 showing additive efficacy (median CI 0.96).

Data are shown in the tables below (Fa=fraction affected).

TABLE 1
Cell line: Ramos
RRID cell accession identifier: CVCL_0597

| ADCx19 | Pola-V | | | ADCx19 | Pola-V | | |
|---|---|---|---|---|---|---|---|
| (pM) | (ng/ml) | Fa | CI | (pM) | (ng/ml) | Fa | CI |
| 0.01 | 250.0 | 0.85 | 0.79 | 0.20 | 3.9 | 0.26 | 1.07 |
| 0.01 | 62.5 | 0.67 | 0.73 | 0.20 | 1.0 | 0.21 | 0.96 |
| 0.01 | 15.6 | 0.18 | 2.75 | 0.20 | 0.2 | 0.26 | 0.71 |
| 0.01 | 3.9 | 0.05 | 3.94 | 0.78 | 250.0 | 0.98 | 0.17 |
| 0.01 | 1.0 | 0.05 | 1.34 | 0.78 | 62.5 | 0.93 | 0.32 |
| 0.01 | 0.2 | 0.07 | 0.28 | 0.78 | 15.6 | 0.87 | 0.44 |
| 0.05 | 250.0 | 0.85 | 0.85 | 0.78 | 3.9 | 0.87 | 0.43 |
| 0.05 | 62.5 | 0.64 | 0.89 | 0.78 | 1.0 | 0.86 | 0.42 |
| 0.05 | 15.6 | 0.16 | 3.74 | 0.78 | 0.2 | 0.88 | 0.38 |
| 0.05 | 3.9 | 0.09 | 2.42 | 3.13 | 250.0 | 0.97 | 0.59 |
| 0.05 | 1.0 | 0.05 | 1.58 | 3.13 | 62.5 | 0.95 | 0.82 |
| 0.05 | 0.2 | 0.10 | 0.45 | 3.13 | 15.6 | 0.96 | 0.75 |
| 0.20 | 250.0 | 0.92 | 0.38 | 3.13 | 3.9 | 0.96 | 0.75 |
| 0.20 | 62.5 | 0.72 | 0.72 | 3.13 | 1.0 | 0.96 | 0.70 |
| 0.20 | 15.6 | 0.28 | 2.02 | 3.13 | 0.2 | 0.97 | 0.60 |

TABLE 2
Cell line: TMD8
RRID cell accession identifier: CVCL_A442
Reference: Tohda et al., Leuk. Res. 30: 1385-1390(2006)

| ADCx19 | Pola-V | | | ADCx19 | Pola-V | | |
|---|---|---|---|---|---|---|---|
| (pM) | (ng/ml) | Fa | CI | (pM) | (ng/ml) | Fa | CI |
| 0.025 | 222 | 0.91 | 0.70 | 6.25 | 8 | 0.17 | 1.09 |
| 0.025 | 74 | 0.70 | 0.66 | 6.25 | 3 | 0.12 | 1.32 |
| 0.025 | 25 | 0.08 | 2.32 | 25 | 222 | 0.97 | 0.27 |
| 0.025 | 8 | 0.01 | 3.87 | 25 | 74 | 0.93 | 0.21 |
| 0.025 | 3 | 0.01 | 1.52 | 25 | 25 | 0.80 | 0.21 |
| 0.1 | 222 | 0.90 | 0.73 | 25 | 8 | 0.61 | 0.30 |
| 0.1 | 74 | 0.68 | 0.71 | 25 | 3 | 0.48 | 0.45 |
| 0.1 | 25 | 0.05 | 3.42 | 25 | 222 | 0.99 | 0.38 |
| 0.1 | 8 | 0.01 | 3.45 | 25 | 74 | 0.93 | 0.48 |
| 0.1 | 3 | 0.01 | 1.74 | 25 | 25 | 0.77 | 0.75 |
| 0.39 | 222 | 0.84 | 1.09 | 25 | 8 | 0.63 | 1.06 |
| 0.39 | 74 | 0.62 | 0.86 | 25 | 3 | 0.57 | 1.22 |
| 0.39 | 25 | 0.07 | 2.93 | 50 | 222 | 0.99 | 0.31 |
| 0.39 | 8 | 0.01 | 5.67 | 50 | 74 | 0.95 | 0.48 |
| 0.39 | 3 | 0.01 | 3.17 | 50 | 25 | 0.86 | 0.80 |
| 1.56 | 222 | 0.90 | 0.74 | 50 | 8 | 0.76 | 1.17 |
| 1.56 | 74 | 0.66 | 0.76 | 50 | 3 | 0.72 | 1.37 |
| 1.56 | 25 | 0.17 | 1.48 | 100 | 222 | 0.98 | 0.23 |
| 1.56 | 8 | 0.05 | 2.23 | 100 | 74 | 0.89 | 0.37 |
| 1.56 | 3 | 0.02 | 3.51 | 100 | 25 | 0.88 | 0.22 |
| 3 | 222 | 0.93 | 0.97 | 100 | 8 | 0.89 | 0.14 |
| 3 | 74 | 0.73 | 0.82 | 100 | 3 | 0.86 | 0.16 |
| 3 | 25 | 0.31 | 1.04 | 100 | 222 | 1.00 | 0.25 |
| 3 | 8 | 0.07 | 2.28 | 100 | 74 | 0.98 | 0.31 |
| 3 | 3 | 0.03 | 4.17 | 100 | 25 | 0.93 | 0.71 |
| 6 | 222 | 0.96 | 0.73 | 100 | 8 | 0.86 | 1.30 |
| 6 | 74 | 0.80 | 0.70 | 100 | 3 | 0.84 | 1.44 |
| 6 | 25 | 0.53 | 0.74 | 200 | 222 | 0.99 | 0.49 |
| 6 | 8 | 0.19 | 1.60 | 200 | 74 | 0.99 | 0.35 |
| 6 | 3 | 0.12 | 2.19 | 200 | 25 | 0.98 | 0.39 |
| 6.25 | 222 | 0.95 | 0.46 | 200 | 8 | 0.96 | 0.83 |
| 6.25 | 74 | 0.84 | 0.37 | 200 | 3 | 0.95 | 0.90 |
| 6.25 | 25 | 0.49 | 0.50 | | | | |

TABLE 3
Cell line: WSU-DLCL2
RRID cell accession identifier: CVCL_1902

| ADCx19 | Pola-V | | | ADCx19 | Pola-V | | |
|---|---|---|---|---|---|---|---|
| (pM) | (ng/ml) | Fa | CI | (pM) | (ng/ml) | Fa | CI |
| 0.0064 | 48.8 | 0.01 | 10.82 | 0.8 | 12.3 | 0.34 | 0.59 |
| 0.0064 | 37.0 | 0.28 | 0.53 | 0.8 | 12.2 | 0.51 | 0.38 |
| 0.0064 | 12.3 | 0.00 | 15.78 | 0.8 | 4.1 | 0.31 | 0.60 |
| 0.0064 | 12.2 | 0.01 | 2.93 | 0.8 | 3.1 | 0.43 | 0.41 |
| 0.0064 | 4.1 | 0.01 | 2.40 | 0.8 | 1.4 | 0.29 | 0.63 |
| 0.0064 | 3.1 | 0.01 | 0.96 | 4 | 48.8 | 0.87 | 0.30 |
| 0.0064 | 1.4 | 0.01 | 0.88 | 4 | 37.0 | 0.83 | 0.23 |
| 0.032 | 48.8 | 0.01 | 12.04 | 4 | 12.3 | 0.80 | 0.24 |
| 0.032 | 37.0 | 0.18 | 0.95 | 4 | 12.2 | 0.83 | 0.31 |
| 0.032 | 12.3 | 0.03 | 2.27 | 4 | 4.1 | 0.76 | 0.30 |
| 0.032 | 12.2 | 0.01 | 4.16 | 4 | 3.1 | 0.80 | 0.33 |
| 0.032 | 4.1 | 0.01 | 3.33 | 4 | 1.4 | 0.74 | 0.32 |
| 0.032 | 3.1 | 0.01 | 2.18 | 20 | 48.8 | 0.88 | 0.96 |
| 0.032 | 1.4 | 0.01 | 2.24 | 20 | 37.0 | 0.88 | 0.56 |
| 0.16 | 48.8 | 0.02 | 9.15 | 20 | 12.3 | 0.87 | 0.64 |
| 0.16 | 37.0 | 0.12 | 1.87 | 20 | 12.2 | 0.86 | 1.07 |
| 0.16 | 12.3 | 0.01 | 13.39 | 20 | 4.1 | 0.85 | 0.74 |
| 0.16 | 12.2 | 0.01 | 10.26 | 20 | 3.1 | 0.87 | 0.95 |
| 0.16 | 4.1 | 0.01 | 10.13 | 20 | 1.4 | 0.84 | 0.82 |
| 0.16 | 3.1 | 0.01 | 8.29 | 100 | 37.0 | 0.91 | 1.95 |
| 0.16 | 1.4 | 0.01 | 9.04 | 100 | 12.3 | 0.90 | 2.21 |
| 0.8 | 48.8 | 0.66 | 0.41 | 100 | 4.1 | 0.89 | 2.36 |
| 0.8 | 37.0 | 0.50 | 0.45 | 100 | 1.4 | 0.88 | 2.56 |

### Example 2: In Vivo Efficacy of a ADCx19 and Pola-V Combination

### Methods

Female severe combined immunodeficient mice (Fox Chase SCID®, CB17/Icr-Prkdc^{scid}/IcrIcoCrl, Charles River) were nine weeks old with a body weight (BW) range of 17.3 to 24.1 g on Day 1 of the study.

On the day of implant, 1×10⁷ WSU-DLCL2 cells (0.1 mL suspension) were subcutaneously implanted into the right flank of each test animal and tumors were monitored as their volumes approached the target range.

Ten days after tumor implantation, designated as Day 1 of the study, the animals were sorted into groups (n=10) with individual tumor volumes of 88 to 126 mm³ and group mean tumor volumes of 109 to 111 mm³.

All vehicle and ADC doses were administered i.v. via tail vein injection once on Day 1 (qd×1). The dosing volume was 0.2 mL per 20 grams of body weight (10 mL/kg) and was scaled to the body weight of each individual animal.

Tumors were measured using calipers twice per week, and each animal was euthanized when its tumor reached the endpoint volume of 1000 mm³ or at the end of the study, whichever came first. The study ended on Day 62.

Tumors were measured in two dimensions using calipers, and volume was calculated using the formula

Tumor Volume (mm³)=w²×l/2, where w=width and l=length, in mm, of the tumor.

Tumor weight may be estimated with the assumption that 1 mg is equivalent to 1 mm³ of tumor volume.

### Results

In this study, Pola-V is the CD79bxADC used and Loncastuximab tesirine is the CD19xADC used.

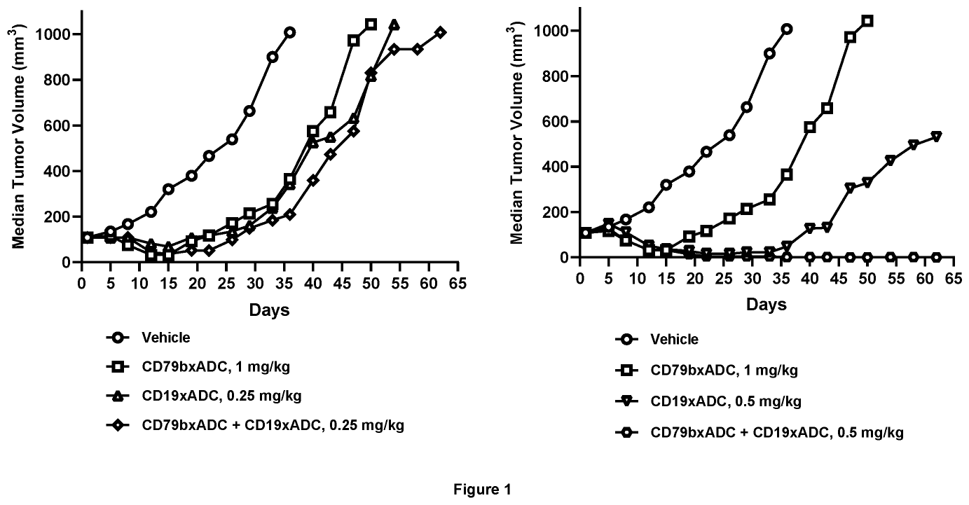

FIG. 1 shows a plot of median tumour volume. The tables below sets out the study response summary, where PR=partial response, CR=complete response, TFS=tumour free survivor.

TABLE 4

| Response summary | PR | CR | TFS |
|---|---|---|---|
| Vehicle | 0 | 0 | 0 |
| CD79bxADC, 1 mg/kg | 3 | 3 | 1 |
| CD19xADC, 0.25 mg/kg | 3 | 0 | 0 |
| CD19xADC, 0.5 mg/kg | 2 | 5 | 3 |
| CD79bxADC, 1 mg/kg + CD19xADC, 0.25 mg/kg | 2 | 3 | 1 |
| CD79bxADC, 1 mg/kg + CD19xADC, 0.5 mg/kg | 1 | 8 | 6 |

### Example 3: In Vivo Efficacy of a ADCx19 and Pola-V Combination

A further in vivo study was conducted, similar to Example 2 but using a Ramos xenograft model.

### Methods

Female severe combined immunodeficient mice (Fox Chase SCID®, CB17/Icr-Prkdc^{scid}/IcrIcoCrl, Charles River) were eight weeks old with a body weight (BW) range of 15.6 to 22.9 g on Day 1 of the study.

On the day of implant, 1×10⁷ Ramos cells (0.1 mL suspension) were subcutaneously implanted into the right flank of each test animal and tumors were monitored as their volumes approached the target range. All vehicle and ADC doses were administered i.v. via tail vein injection once on Day 1 (qd×1). The dosing volume was 0.2 mL per 20 grams of body weight (10 mL/kg) and was scaled to the body weight of each individual animal.

Fourteen days after tumor implantation, designated as Day 1 of the study, the animals were sorted into groups (n=10) with individual tumor volumes of 108 to 144 mm³ and group mean tumor volumes of 130 to 134 mm³.

Tumors were measured using calipers twice per week, and each animal was euthanized when its tumor reached the endpoint volume of 1000 mm³ or at the end of the study, whichever came first. The study ended on Day 62.

### Results

TABLE 5

| Response summary | PR | CR | TFS |
|---|---|---|---|
| Vehicle | 0 | 0 | 0 |
| CD79bxADC, 2 mg/kg | 0 | 0 | 0 |
| CD19xADC, 0.3 mg/kg | 0 | 0 | 0 |
| CD19xADC, 0.6 mg/kg | 0 | 7 | 7 |
| CD79bxADC, 2 mg/kg + CD19xADC, 0.3 mg/kg | 1 | 0 | 0 |
| CD79bxADC, 2 mg/kg + CD19xADC, 0.6 mg/kg | 0 | 10 | 9 |

In vivo, combination of CD19xADC with CD79bxADC resulted in improved anti-tumor activity and better response rates in the WSU-DLCL2 and Ramos xenograft models. The combination was acceptably well tolerated in both models. Translation of these pre-clinical data in the clinic is currently being investigated in a phase I trial evaluating the safety and tolerability of loncastuximab tesirine in combination with polatuzumab vedotin in patients with r/r NHL (NCT04970901).

### Example 4: In Vitro Efficacy of SG3199 (PBD) and MMAE Combination

We tested a combination of the warheads alone used in CD19xADC (PBD SG3199) and CD79bxADC (monomethyl auristatin E (MMAE)). This was performed essentially as per Example 1, in TMD8 cells. Cells were seeded at a density of 50,000 cells/mL. We used SG3199 and MMAE with the following titration: SG3199—a 7 point 1 in 4 serial titration from 230 pM to 0.06 pM; MMAE: a 7 point 1 to 3 serial titration from 50 nM to 0.08 nM.

Cells were incubated for 24 hours and then viability was measured with CellTiterGlo assay.

For analysis of the results, data were first blank corrected (average of blank of the 3 replicates was subtracted from each data value). For GraphPad analysis, each replicate was normalized over the untreated cells (average of the 3 replicates). Data were analysed by GraphPad Prism to generate a dose response curve with Log drug concentration on X-axis and cell viability (%) on Y-axis. For CalcuSyn analysis, the average of the 3 replicates was corrected over the untreated cells.

The median Chou-Talalay combination index (CI) was 0.85 (<0.9 indicates synergy).

TABLE 6

| SG3199 | MMAE | | | SG3199 | MMAE | | |
|---|---|---|---|---|---|---|---|
| (pM) | (ng/ml) | Fa | CI | (pM) | (ng/ml) | Fa | CI |
| 0.06 | 0 | 0.12 | N/A | 0.06 | 0.08 | 0.12 | 1.65 |
| 0.23 | 0 | 0.16 | N/A | 0.23 | 0.08 | 0.05 | 434.19 |
| 0.90 | 0 | 0.15 | N/A | 0.90 | 0.08 | 0.19 | 0.58 |
| 3.6 | 0 | 0.15 | N/A | 3.6 | 0.08 | 0.16 | 5.16 |
| 14.4 | 0 | 0.19 | N/A | 14.4 | 0.08 | 0.17 | 12.16 |
| 57.5 | 0 | 0.23 | N/A | 57.5 | 0.08 | 0.34 | 0.22 |
| 230 | 0 | 0.46 | N/A | 230 | 0.08 | 0.47 | 0.03 |
| 0 | 0.08 | 0.11 | N/A | 0.06 | 0.2 | 0.13 | 2.06 |
| 0 | 0.2 | 0.17 | N/A | 0.23 | 0.2 | 0.14 | 2.22 |
| 0 | 0.6 | 0.27 | N/A | 0.90 | 0.2 | 0.16 | 2.08 |
| 0 | 1.9 | 0.29 | N/A | 3.6 | 0.2 | 0.11 | 69.07 |
| 0 | 5.6 | 0.38 | N/A | 14.4 | 0.2 | 0.16 | 18.45 |
| | | | | 57.5 | 0.2 | 0.33 | 0.39 |
| | | | | 230 | 0.2 | 0.51 | 0.02 |
| 0.06 | 0.6 | 0.27 | 0.53 | 0.06 | 5.6 | 0.38 | 1.23 |
| 0.23 | 0.6 | 0.29 | 0.40 | 0.23 | 5.6 | 0.40 | 0.98 |
| 0.90 | 0.6 | 0.31 | 0.29 | 0.90 | 5.6 | 0.41 | 0.85 |
| 3.6 | 0.6 | 0.23 | 1.26 | 3.6 | 5.6 | 0.34 | 1.93 |
| 14.4 | 0.6 | 0.29 | 0.60 | 14.4 | 5.6 | 0.39 | 1.12 |
| 57.5 | 0.6 | 0.37 | 0.23 | 57.5 | 5.6 | 0.49 | 0.37 |
| 230 | 0.6 | 0.54 | 0.03 | 230 | 5.6 | 0.62 | 0.09 |
| 0.06 | 1.9 | 0.32 | 0.79 | | | | |
| 0.23 | 1.9 | 0.29 | 1.18 | | | | |
| 0.90 | 1.9 | 0.32 | 0.83 | | | | |
| 3.6 | 1.9 | 0.28 | 1.36 | | | | |
| 14.4 | 1.9 | 0.29 | 1.32 | | | | |
| 57.5 | 1.9 | 0.42 | 0.30 | | | | |
| 230 | 1.9 | 0.59 | 0.04 | | | | |

### Example 5: In Vitro Efficacy of an ADCx25 and Pola-V Combination

### Methods

MTT proliferation assay on cell lines to be exposed (120 h) to increasing ADCx25 and Pola-V concentrations. Synergy at 120 h will be assessed by Chou-Talalay combination index (CI) (synergism CI<0.9, additive CI=0.9-1.1, antagonism/no benefit CI>1.1).

### Example 6: In Vivo Efficacy of an ADCx25 and Pola-V Combination

### Methods

Female severe combined immunodeficient mice (Fox Chase SCID®, CB17/Icr-Prkdc^{scid}/IcrIcoCrl, Charles River) will be nine weeks old with a body weight (BW) range of 17.3 to 24.1 g on Day 1 of the study.

The study will be further conducted as per Examples 2 and 3.

### Example 7: In Vitro Efficacy of an ADCx22 and Pola-V Combination

### Methods

MTT proliferation assay on cell lines to be exposed (120 h) to increasing ADCx22 and Pola-V concentrations. Synergy at 120 h will be assessed by Chou-Talalay combination index (CI) (synergism CI<0.9, additive CI=0.9-1.1, antagonism/no benefit CI>1.1) on a germinal center (GCB) DLBCL (WSU-DLCL2) cell line and a Burkitt lymphoma (Ramos) cell line.

### Example 8: In Vivo Efficacy of an ADCx22 and Pola-V Combination

### Methods

Female severe combined immunodeficient mice (Fox Chase SCID®, CB17/Icr-Prkdc^{scid}/IcrIcoCrl, Charles River) will be subcutaneously implanted into the right flank of each test animal with either WSU-DLCL2 or Ramos cells and tumors will be monitored as their volumes approached the target range.

The study will be further conducted as per Examples 2 and 3.

A number of publications are cited above to more fully describe and disclose the disclosures and the state of the art to which inventions herein may pertain. The entirety of each of the references mentioned in this disclosure is hereby incorporated by reference.

1-24. (canceled)

25. A method for treating a proliferative disorder in an individual, the method comprising administering to the individual an effective amount of an antibody drug conjugate (ADC) comprising an anti-CD19 antibody and a pyrrolobenzodiazepine (PBD) dimer and polatuzumab vedotin, wherein the PBD dimer is of formula (III): 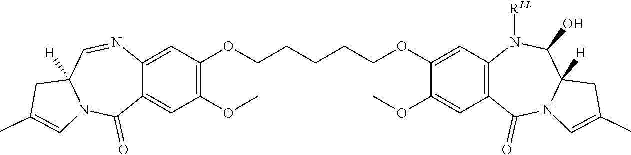
wherein R^{LL} is a linker for connection to the antibody.

26. The method according to claim 25, wherein the ADC has the following structure: 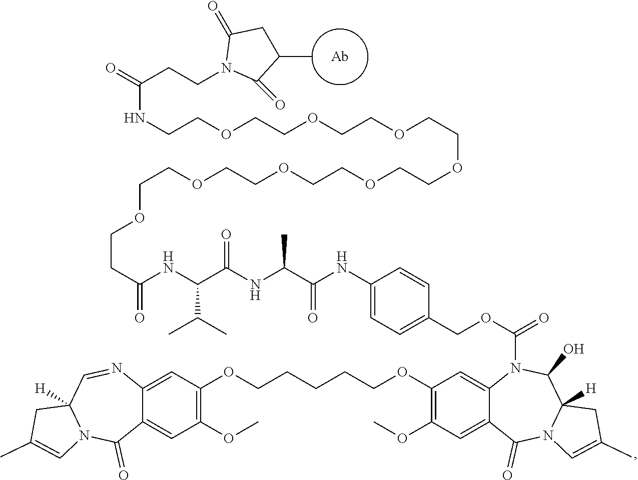
wherein Ab is the anti-CD19 antibody.

27. The method according to claim 25, wherein the anti-CD19 antibody comprises the complementarity-determining regions (CDRs) of SEQ ID NOs: 2 and 8.

28. The method according to claim 25, wherein the individual is human.

29. The method according to claim 25, wherein the individual has, or has been determined to have, a cancer which expresses CD19 or CD19+ve tumour-associated non-tumour cells.

30. The method according to claim 29, wherein the CD19+ve tumour-associated non-tumour cells are CD19+ve infiltrating cells.

31. The method according to claim 25, wherein the individual is undergoing treatment with polatuzumab vedotin.

32. The method according to claim 25, wherein the individual has undergone treatment with polatuzumab vedotin.

33. The method according to claim 25, wherein the individual is refractory to treatment, or further treatment, with polatuzumab vedotin.

34. The method according to claim 25, wherein the treatment with the ADC and polatuzumab vedotin has increased efficacy as compared to monotherapy with either the ADC or polatuzumab vedotin alone.

35. The method according to claim 25, wherein the disorder is characterised by the presence of a neoplasm comprising CD19+ve cells.

36. The method according to claim 25, wherein the disorder is cancer.

37. The method according to claim 25, wherein the disorder is selected from the group comprising: non-Hodgkin's Lymphoma, including diffuse large B-cell lymphoma (DLBCL), follicular lymphoma, (FL), Burkitt lymphoma, Mantle Cell lymphoma (MCL), chronic lymphatic lymphoma (CLL), Waldenström Macroglobulinemia (WM), and Marginal Zone B-cell lymphoma (MZBL), and leukemias such as Hairy cell leukemia (HCL), Hairy cell leukemia variant (HCL-v), chronic lymphocytic leukemia (CLL) including Richter syndrome, and Acute Lymphoblastic Leukaemia (ALL) such as Philadelphia chromosome-positive ALL (Ph+ALL) or Philadelphia chromosome-negative ALL (Ph-ALL).

38. A composition comprising (i) an antibody drug conjugate (ADC) comprising an anti-CD19 antibody and a pyrrolobenzodiazepine (PBD) dimer, and (ii) polatuzumab vedotin, wherein the PBD dimer is of formula (III): 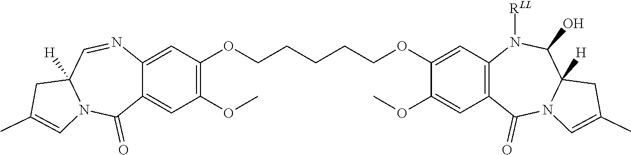
wherein R^{LL} is a linker for connection to the antibody.

39. The composition according to claim 38, wherein the ADC has the following structure: 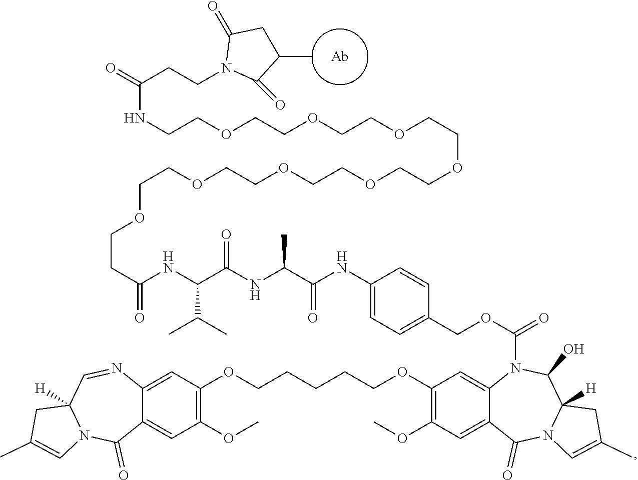
wherein Ab is the anti-CD19 antibody.

40. The composition according to claim 38, wherein the anti-CD19 antibody comprises the complementarity-determining regions (CDRs) of SEQ ID NOs: 2 and 8.

41. A method of selecting an individual for treatment with an anti-CD19 antibody drug conjugate (ADC) comprising an anti-CD19 antibody and a pyrrolobenzodiazepine (PBD) dimer, wherein the individual is selected for treatment with the ADC if the individual has been treated or is being treated with polatuzumab vedotin.
